(19) United States
(12) Patent Application Publication
(10) Pub. No.: US 2013/0218005 A1
(43) Pub. Date: Aug. 22, 2013

(54) MINIMALLY INVASIVE NEUROSURGICAL INTRACRANIAL ROBOT SYSTEM AND METHOD

(71) Applicants: UNIVERSITY OF MARYLAND, COLLEGE PARK, (US); UNIVERSITY OF MARYLAND, BALTIMORE, (US)

(72) Inventors: JAYDEV P. DESAI, BETHESDA, MD (US); MINGYEN HO, ADELPHI, MD (US); J. MARC SIMARD, BALTIMORE, MD (US); RAO GULLAPALLI, ELLICOTT CITY, MD (US)

(73) Assignees: UNIVERSITY OF MARYLAND, BALTIMORE, BALTIMORE, MD (US); UNIVERSITY OF MARYLAND, COLLEGE PARK, COLLEGE PARK, MD (US)

(21) Appl. No.: 13/763,284

(22) Filed: Feb. 8, 2013

Related U.S. Application Data
(60) Provisional application No. 61/596,603, filed on Feb. 8, 2012.

(51) Int. Cl. A61B 19/00; A61M 1/00; A61B 18/12; A61B 18/04; A61B 8/08; A61N 7/00; A61B 5/055; A61B 5/06; A61B 6/03; A61B 17/00; A61B 18/20

(52) U.S. Cl. CPC A61B 19/2203; A61B 17/00234; A61M 1/0058; A61B 18/12; A61B 18/042; A61B 18/20; A61N 7/00; A61B 5/055; A61B 5/062; A61B 6/032; A61B 8/0841
USPC 600/424

(57) ABSTRACT

Minimally invasive neurosurgical intracranial robot system is introduced to the operative site by a neurosurgeon through a narrow surgical corridor. The robot is passed through a cannula and is attached to the cannula by a latching mechanism. The robot has several links interconnected via revolute joints which are tendon-driven by tendons routed through channels formed in the walls of the links. The robot is teleoperatively guided by the neurosurgeon based on real-time images of the intracranial operative site and tracking information of the robot position. The robot body is equipped with a tracking system, tissue liquefacting end-effector, at as well as irrigation and suction tubes. Actuators for the tendon-driven mechanism are positioned at a distance from the imaging system to minimize distortion to the images. The tendon-actuated navigation of the robot permits an independent control of the revolute joints in the robot body.

### REFERENCE TO RELATED APPLICATIONS

This Utility patent application is based on the Provisional Patent Application No. 61/596,603 filed on 8 Feb. 2012.

### STATEMENT REGARDING FEDERAL RESPONSIVE RESEARCH OR DEVELOPMENT

The development of the invention described herein was funded by NIH under Grant No. R21EB008796. The U.S. Government has certain rights in this invention.

### FIELD OF THE INVENTION

The present invention relates to minimally invasive surgical procedures, and more in particular to a robotic system for minimally invasive neurosurgery.

Even more in particular, the present invention relates to a high dexterity robot for applications in neurosurgery, which can be teleoperatively controlled by a neurosurgeon to remove deep intracranial tumors, both neoplastic and non-neoplastic masses, such as blood clots.

The present invention further relates to a miniature robot teleoperatively guided by a neurosurgeon based on the images of the intracranial operation site acquired substantially in real-time from an imaging system and the visual data acquired through a tracking system which enables the localization of the end-effector of the subject robot.

In overall concept, the present invention pertains to a miniature robot introduced to an operative site through a flexible cannula inserted by a neurosurgeon through a narrow surgical corridor and attached to the distal end of the cannula (at a desired depth into the operative site) through a latching mechanism formed thereat. The miniature robot is formed by a number of links connected each to an adjacent one through a revolute joint, thus attaining a number of degrees of freedom and out-of-plane motion when steered through a tendon-driven mechanism integrated with the robot body in a manner permitting efficient and independent control of the revolute joints.

The present invention further is related to a miniature robot navigated teleoperatively by a neurosurgeon to reach a tumor, and integrated with an image-guided tracking system including, but not limited to MRI for localization of the robot position. The robot is provided with suction and irrigation channels within the robot structure and treatment tools for tissue removal including, but not limited to one or more of the following: monopolar electrocautery, bi-polar electrocautery, laser ablation, radio-frequency ablation, ultrasonic cavitator, APC (Argon-Plasma Coagulation), etc., that can be employed in various imaging scenarios include, but not limited to MR imaging.

The present invention uniquely combines the tracking ability of the Endoscout® from Robin Medical Systems to provide information on the anchor point of the robot (base) and the information from the tip of the robot to provide real-time imaging information to guide the robot to the appropriate destination.

The present invention is further directed to a miniature robot composed of a number of links fabricated from a material compatible with an imaging technique and interconnected through revolute joints which are placed orthogonally each with respect to the other to provide out-of-plane motion capability. Each revolute joint is tendon-driven independently by an actuator mechanism controlled in correspondence with a neurosurgeon's commands.

Additionally, the present invention relates to a minimally invasive neurosurgical intracranial robot which may be used with a number of imaging techniques including, but not limited to, MRI (Magnetic Resonance Imaging), computer tomography (CT), ultrasound, etc., provided the components of the robotic system are compatible with the particular imaging modality.

In addition, the present invention relates to a minimally invasive neurosurgical robotic system tendon-driven by a controllable actuator mechanism, which is away from the imaging plane of the imaging system to reduce or completely eliminate image distortion.

### BACKGROUND OF THE INVENTION

Brain tumors are among the most deadly adult tumors which accounts for about 2% of all cancer deaths in the United States. The primary reason for the high mortality rate includes the inability to remove the complete tumor tissue due to the location of the tumor deep in the brain, as well as the lack of a satisfactory continuous imaging modality for intraoperative intracranial procedures.

Surgical resection of the tumor is considered the optimal treatment for most brain tumors. To minimize the trauma to the surrounding brain tissues during surgical resection, endoscopic port surgery (EPS) was developed which is a minimally-invasive technique for brain tumor resection which minimizes tissue disruption during tumor removal.

However, due to the lack of satisfactory continuous imaging modality, it is extremely challenging to remove brain tumors precisely and completely without damaging the surrounding brain tissue using traditional surgical tools. As a result, patients may develop hemi paresis, cognitive impairment, stroke or other neurological deficits due to the procedure.

MRI (Magnetic Resonance Imaging) provides excellent soft-tissue contrast which enables the neurosurgeon to perform the procedure with less trauma to surrounding tissues during tumor resection. However, due to the strong magnetic field required in the MRI, commonly used sensors and actuators in conventional robotic systems are precluded from being used in MRI-compatible robots.

Several MRI-compatible surgical robotic systems have been designed in recent years. For example, Masamune, et al. (“Development of an MRI-compatible needle insertion manipulator for stereotactic neurosurgery”, J. of Image Guided Surg., 1995, 1(4), pp. 242-248) developed a MRI-compatible needle insertion manipulator dedicated to neurosurgical applications using ultrasonic motors; Wang, et al. (“MRI compatibility evaluation of a piezoelectric actuator system for a neural interventional robot”, In Proc. IEEE Eng. Med. Biol. Soc. Annu. Int. Conf., 2009, pp. 6072-6075) built an MRI-compatible neural interventional robot using a piezoelectric actuator system.

Kokes, et al. (“Towards a teleoperated needle driver robot with haptic feedback for RFA of breast tumors under continuous MRI”, Med. Image Anal., 2009, 13(3), pp. 445-455) developed an MRI-compatible needle driver system for Radio Frequency Ablation (RFA) of breast tumors using hydraulic actuation.

Yang, et al. (“Design and control of a 1-DOF MRI-compatible pneumatically actuated robot with long transmission lines”, IEEE/ASME Trans. Mechatron., 2011, 16, pp. 1040-1048) presented a design and control of an MRI-compatible 1-DOF needle-driver robot using pneumatic actuation with long transmission lines.

Fischer, et al. (“MRI-compatible pneumatic robot for transperineal prostate needle placement”, IEEE/ASME Trans. Mechatron., 2008, 13(3), pp. 295-305) developed an MRI-compatible robot for prostate needle placement using pneumatic actuation.

Krieger, et al. (“Design of a novel MRI compatible manipulator for image guided prostate interventions”, IEEE Trans. Biomed. Eng., 2005, 52(2), pp. 306-313, and “Development and preliminary evaluation of an actuated MRI-compatible robotic device for MRI-guided prostate intervention”, In Proc. IEEE Int. Conf. Robot. Autom., 2010, pp. 1066-1073) developed an MRI-guided manipulator for prostate interventions using shaft transmission and piezo-ceramic motors.

Although the above-mentioned robotic systems are MRI compatible, they unfortunately cannot be used to reach a target which is not in the “line-of-sight” due to limited Degrees Of Freedom (DOF) of the robots intended for use in their systems.

N. Pappafotis, et al. (“Towards design and fabrication of a miniature MRI-compatible robot for applications in neurosurgery”, in Int. Design Eng. Technical Conf. & Computers and Information in Eng. Conf., 2008) described a preliminary prototype of Minimally Invasive Neurosurgical Intracranial Robot (MINIR) using Shape Memory Alloy (SMA) wires as actuators.

An improved design of MINIR was proposed by Ho, M. and Desai, J. P. (“Towards a MRI-compatible meso-scale SMA-actuated robot using PWM control”, in Int. Conf. on Biomedical Robotics and Biomechatronics, 2010, pp. 361-366) which improved several limitations of previous prototypes. The improved MINIR had individual SMA actuators for each joint. All joints were located on the outside surface of the robot and all wiring and tubes were routed inside the robot, thus attaining a more compact and easier shielded robot.

Shape memory alloy (SMA) based actuators have been widely used in robotic systems and medical devices. The advantages of SMA actuators include large energy density, large stroke, light weight and they can be used directly without additional mechanisms. K. Ikuta, et al. (“Shape memory alloy servo actuator system with electric resistance feedback and application for active endoscope,” in Proc. IEEE Int. Conf Robot. Autom., 1988, pp. 427-430) used SMA tubes to actuate active forceps for laparoscopic surgery. E. Ayvali, et al. (“Towards a discretely actuated steerable cannula for diagnostic therapeutic procedures,” Int. J. Robot. Res., 2012, Vol. 31, No. 5, pp. 588-603,) developed a multi-degree-of-freedom (multi-DOF) discretely actuated steerable cannula using SMA wires as actuators.

SMA has been tested in 1.5-T and 4.1-T MRI scanners and only minor artifact was observed in the MR images as was reported in A. Holton, et al., “Comparative MRI compatibility of 316L stainless steel alloy and nickel-titanium alloy stents,” J. of Cardiovascular Magnetic Resonance, 2002, Vol. 4., No. 4, pp. 423-430.

M. Ho, et al. (“Towards a MR image-guided SMA-actuated neurosurgical robot,” in Proc, IEEE Int. Conf Robot, Autom., 2011, pp. 1153-1158; and “Toward a meso-scale SMA-actuated MRI-compatible neurosurgical robot,” IEEE Trans. Robot., 2012, Vol. 28, No. 1, pp. 213-222), presented an MRI-compatible minimally invasive neurosurgical intracranial robot (MINIR) using SMA wires as actuators.

In M. Ho, et al. (“Towards a MR image-guided SMA-actuated neurosurgical robot”, in proceedings of 2011 IEEE Int. Con. On Robotics and Automation, 2011, pp. 1153-1158), the force behavior of SMA (Shape Memory Alloy) actuators in the bent configurations was investigated. In addition, it was demonstrated that vision-based control can be used to precisely control the motion of MINIR.

Though the approach of using SMA (Shape Memory Alloy) wires as actuators was successful, there are significant limitations. Specifically, heating current has to be applied to the SMA wires while actuating the robot. The current can interfere with the magnetic field inside the MRI bore, and thus may lead to some distortion in the image. Although the effects are limited and the profile of MINIR can be easily identified in the MR images, as presented in M. Ho, et al. (“Towards a MR image-guided SMA-actuated neurosurgical robot”, in proceedings of 2011 IEEE Int. Con. On Robotics and Automation, 2011, pp. 1153-1158), the noise and distortion might still cause difficulties finding precise tumor boundaries.

It is clear that an improved MINIR system is needed in which MRI noise would be eliminated.

### SUMMARY OF THE INVENTION

It is therefore an object of the present invention to provide a robotic system for minimally invasive surgical procedures which can be teleoperatively controlled by a neurosurgeon in a highly efficient and precise manner.

It is another object of the present invention to provide a minimally invasive neurosurgical intracranial robot highly compatible with an imaging modality used for the surgery and which is further teleoperatively navigated by a neurosurgeon in an intraoperative imaging modality environment based on the visual information available to the neurosurgeon in the form of frequently updated images of the operation area of interest.

It is a further object of the present invention to provide a minimally invasive intracranial neurosurgery system fully compatible with the MRI (Magnetic Resonance Imaging) technique where the MRI noise and distortion may be eliminated due to the use of a tendon-sheath mechanism adapted for controllable navigation of the robot at the intracranial operational site, thereby attaining acquisition of precise boundaries of tumors, both neoplastic and non-neoplastic masses, such as blood clots, which is highly beneficial for a successful surgical procedure.

It is another object of the present invention to provide a minimally invasive neurosurgical intracranial robot which is introduced to the operative site through a flexible cannula inserted by a neurosurgeon through a narrow surgical corridor and which is teleoperatively steered by the surgeon by controlling the tendon-driven mechanism integrated in the robot body in the most ergonomical and compact manner based on the real-time images obtained on a screen of the neurosurgeon's interface.

It is a further object of the present invention to provide a minimally invasive neurosurgical intracranial robot composed of a plurality of links interconnected each with the other through revolute joints where the adjacent revolute joints are positioned orthogonally each with respect to the other for out-of-plane motion with a large number of degrees-of-freedom. Each revolute joint is controlled independently through a tendon-driven mechanism integrated with the robot body, with the tendons passing in the channels formed in walls of the links.

It is an additional object of the present invention to provide a minimally invasive neurosurgical intracranial robot system where actuators (such as SMA spring actuators, motors, etc.) are positionally displaced from the imaging region of an MRI scanner and exert a required torque at each revolute joint of the robot body through the tendon-driven mechanism.

It is still another object of the present invention to provide a minimally invasive neurosurgical intracranial robot system integrated with a tracking system, a mechanism for tissue removal, and suction and irrigation tubes routed through the hollow robot body.

In one aspect thereof, the present invention constitutes a Minimally Invasive Neurosurgical Intracranial Robot (MINIR) system, which includes:

a robot sub-system compatible with an imaging system used in the surgical procedure. The robot sub-system is introduced in an intracranial area for remote control by a neurosurgeon;

an image tracking and guidance sub-system which is operatively coupled to the robot sub-system, interfaces with the imaging sub-system, and generates tracking information corresponding to the robot sub-system position; and

an interface operatively coupled to the imaging sub-system and the tracking sub-system displays the substantially real-time images aligned with the tracking information.

The interface is operatively interconnected between the neurosurgeon and the robot sub-system to provide the neurosurgeon with a tool to remotely manipulate the robot sub-system based on real-time images and tracking information in order to reach the target of interest (tumor) for an intended tissue liquefaction procedure.

The robot sub-system includes:

a robot body composed of a plurality of links and N revolute joints interconnecting adjacent links each to the other. Each of N revolute joints is formed between respective adjacent links for rotational motion of each link with respect to the other about a corresponding rotational axis extending through each revolute joint in substantially orthogonal relationship to a rotational axis of an adjacent revolute joint.

The robot sub-system further includes a tendon sub-system integrated with the robot body and containing N independent tendons routed through walls of the links, wherein each of the N tendons is operatively coupled to a respective one of the N revolute joints.

An actuator sub-system is operatively coupled to the tendon sub-system. The actuator sub-system contains N independently operated actuating mechanisms. Each actuating mechanism is operatively coupled to a respective one of the N revolute joints through a respective one of the N tendons. Each actuating mechanism independently controls a respective revolute joint by controlling the motion of a respective tendon of the tendon sub-system.

A control sub-system is operatively coupled between the neurosurgeon's interface and the actuator sub-system. The control sub-system generates control signals responsive to the neurosurgeon's commands entered via the interface and transmits the control signals to the actuator sub-system. The actuator sub-system, responsive to the control signals received thereat, controls, by controlling motion of at least one respective tendon, the rotational displacement of adjacent links of one or more revolute joints, thereby steering the robot sub-system relative to the target of interest.

The plurality of links include a tip link, a base link, and intermediate links interconnected between the tip and base links. The tip link carries an end-effector attached thereto.

An irrigation channel extends internally through the robot body between the tip link and an external irrigation hardware, where one end of the irrigation channel interacts with the intracranial area. Additionally, a suction channel extends internally through the hollow robot body between the tip link and external suction hardware. One end of the suction channel extends for interaction with the intracranial area.

The end-effector is adapted for intended interaction with the tumors, both neoplastic and non-neoplastic masses, such as blood clots. Preferably, the end-effector is electrically coupled to end-effector hardware through wiring extending internally of the hollow robot body.

The end-effector member is adapted for a tissue liquefaction of the tumor, and may be selected from a number of techniques, including but not limited to one or more of the following: monopolar electrocautery, bi-polar electrocautery, APC (Argon-Plasma Coagulation), laser ablation, radio-frequency ablation, ultrasonic cavitation, etc.

The robot sub-system further is equipped with a flexible cannula insertable in a surgical corridor towards the intracranial area. The cannula is configured to permit passage of the robot body therethrough.

The cannula is formed with a latching mechanism positioned at an internal wall at the distal end thereof. The latching mechanism is engageably compatible with the base link of the robot body to provide securement of the base link to the cannula at its distal end.

The latching mechanism may include a plurality of latches arranged circumferentially at the internal wall of the cannula at various distances from an edge of the cannula to permit adjustment of the depth to which the robot body may be introduced into the brain.

The tracking sub-system may comprise an Endoscout® tracking system integrated with the robot sub-system and operatively coupled to the interface. The Endoscout® tracking system includes a first Endoscout® sensor integrated with the robot body and positioned at the tip link. An Endoscout® data processing unit is positioned externally of the intracranial area with Endoscout® wiring extending internally through the robot body between the first Endoscout® sensor and the Endoscout® data processing unit positioned outside the imaging site. The Endoscout® tracking system further includes a second Endoscout® sensor positioned at a distal end of the cannula.

The tracking sub-system will utilize unique MR pulse sequences tailored for the MINIR system to generate reliable real-time tracking coordinates for feed-back to scanner sub-system for obtaining images in the desired plane. This information may be used for tracking or to obtain high-resolution images.

The imaging system may be selected from a number of imaging technologies, and may include Magnetic Resonance Imaging (MRI) system, Computed Tomography (CT) imagining system, an Ultrasound imaging system, etc.

The actuator sub-system may include N independently controlled SMA (Shape Memory Alloy) spring actuators. Each spring actuator is operatively coupled to the respective revolute joint via a respective independent tendon of the tendon sub-system. Preferably, each SMA spring actuator includes antagonistically coupled SMA springs.

The real-time imaging feedback system may be selected to perform temperature mapping of the tissue during electrocautery when necessary.

Alternatively, the actuator sub-system includes N motors, each operatively coupled to a respective one of the N revolute joints. In this implementation, the control signals are applied to at least one of the N motors to actuate them for controlling the motion of at least one tendon in the tendon sub-systems.

The robot sub-system is equipped with a visual feedback control between the Endoscout® sensors and the control sub-system. The visual feedback control is based on the tracking information acquired from the tracking system.

In addition, the system is provided with a temperature based feedback sub-system coupled between the SMA springs and the control sub-system. The temperature based feedback sub-system acquires data on the temperature regime applied to a respective SMA spring and a corresponding rotational angle of a revolute joint affected by the respective SMA spring.

Alternatively, the temperature feedback scheme may be replaced by a feedback mechanism based on readings of the rotary encoders if the actuator sub-system uses motors as actuating mechanisms.

The system further is provided with a first set of N gears/pulleys secured in the base link. Each of the pulleys carries therearound a respective one of the N tendons of the tendon sub-system. The system further includes a second set of N gears/pulleys removeably coupled with the gears in the first set.

The actuator sub-system is positioned remotely from the imaging system and operatively coupled thereto through an intermediate quick-connect mechanism having the second set of gears/pulleys at one end and a third set of N gears/pulleys positioned at another end of the intermediate quick-connect mechanism.

The intermediate tendon sub-system including N intermediate tendons is coupled between the second set of the gears/pulleys and a respective one of the N actuating mechanisms to independently control motion of a corresponding tendon of the tendon sub-system, thereby controllably steering the robot sub-system.

The control sub-system further includes a data transformation unit receiving, at an input thereof, the neurosurgeon's commands. It calculates corresponding control signals based on the position and configuration of the robot body. The control signals include coordinates of one (or several) center (centers) of rotation, i.e., at least one revolute joint, and tracking path interpolation. The control signals are operatively coupled to the actuator sub-system to control the robot body's tendon sub-system.

In another aspect, the present invention is directed to method for minimally invasive intracranial neurosurgery. The subject method comprises the steps of:

forming a surgical path towards an intracranial area containing a target of interest which may be a tumor, both neoplastic and non-neoplastic masses, such as blood clots; and

introducing a Minimally Invasive Neurosurgical Intracranial Robot (MINIR) device to the intracranial area through the surgical path.

The MINIR device includes a robot body composed of a plurality of links interconnected at N revolute joints, where each of the N revolute joints is formed between respective adjacent links for rotational motion of each link with respect to the other about a corresponding rotational axis extending through each revolute joint in substantially orthogonal relationship to a rotational axis of an adjacent revolute joint.

A tendon sub-system is integrated with the robot body and contains N independent tendons routed through walls of the links in a predetermined order, wherein each tendon is operatively coupled to a respective revolute joint.

A tracking sub-system is integrated with the robot body. The tracking system has at least one sensor which is positioned in proximity to the end tip of the robot body to generate information corresponding to a position of the tip of the robot body or any other point of interest on the robot body.

The method continues with the steps of:

obtaining, substantially in real-time, images of the intracranial area containing the target of interest (tumor) on a display of an neurosurgeon's interface;

aligning the tracking information (in the form of robot's coordinates) acquired from the tracking sub-system and the real-time images of the intracranial area on the display of the operator's interface; and

prompting the neurosurgeon, through the interface, to control the robot body position and configuration based on the tracking information and the real-time images by entering the neurosurgeon's commands via the interface.

Responsive to the neurosurgeon's commands, the procedure continues through the steps of:

calculating and operatively applying control signals to the tendon sub-system to control rotational motion at one or more respective revolute joints by controlling motion of at least one tendon in the tendon sub-system coupled to the respective revolute joint, thereby navigating the robot body relative to the target of interest.

The subject method further continues through the steps of:

operatively coupling a control sub-system between the tendon sub-system and the interface;

coupling an actuator sub-system between the control sub-system and the tendon sub-system, where the actuator sub-system includes N actuating mechanisms, each operatively coupled to a respective one of N independent tendons in the tendon sub-system; and

controlling the rotational motion of the adjacent links at a respective revolute joint by controlling motion of the respective independent tendon by a respective actuating mechanism in correspondence to the control signals applied to the respective actuator mechanism.

Preferably, the method is enhanced through the steps of:

inserting a flexible cannula in the surgical corridor, where the cannula is configured to permit passage of the robot body therethrough,

introducing the MINIR device to the intracranial area through the cannula, and

securing the robot body at a distal end of the cannula by a latching mechanism provided thereat.

The present method attains high preciseness and efficiency of the surgical procedure through the steps of:

carrying out a visual feedback control between the tracking sub-systems and the control sub-system, and, in addition, using a temperature-based feedback control when the SMA springs are used as part of the actuator sub-system.

Further operations of the subject method are carried out through the steps of:

receiving, at an input of the control sub-system, the neurosurgeon's commands, and

computing corresponding control signals based on the position and configuration of the robot body, where the control signals include coordinates of the center of rotation at the robot body and tracking path interpolation, and

applying the control signals to the actuator sub-system to control motion of at least one corresponding tendon in the tendon sub-system.

During the surgery, a neurosurgeon may obtain high-resolution diagnostic quality images of the intracranial area when needed.

These and other features and advantages of the present invention will be apparent from the following detailed description taken in conjunction with accompanying patent drawings.

### BRIEF DESCRIPTION OF THE DRAWINGS

FIG. 1 is a block diagram representing, in somewhat simplified form, the surgical setup using the minimally invasive neurosurgical intracranial robot system of the present invention;

FIG. 2A is a pictorial view of the robot sub-system and actuator sub-system interconnected through the intermediate quick-connect tendon-sheath mechanism of the present invention;

FIG. 2B is a representation of the robot sub-system of the present invention introduced into the operative site through a surgical corridor;

FIG. 3 is a pictorial representation of the MINIR latched at the end of a cannula;

FIG. 4A is a cross-sectional view of the distal end of the cannula showing a latching mechanism at the interior of the cannula;

FIG. 4B illustrates the operation of the latching mechanism at the distal end of the cannula and attachment of the base link of the robot body between two layers of the latching tabs;

FIG. 5 illustrates the MINIR body operatively coupled to the actuator sub-system designed with motor actuators;

FIG. 6 is a schematic representation of the MINIR in its reference configuration;

FIG. 7 is a diagram representative of the workspace of the MINIR body;

FIG. 8A illustrates the operational principles of the actuator system based on SMA antagonistic springs;

FIG. 8B is a schematic representation of the tendon-driven robot steerable by the SMA spring based actuation mechanism;

FIG. 8C is a representation of the contents of the actuator box including SMA spring actuating mechanisms attached to the hardware routing box;

FIG. 8D shows a prototype of the MINIR actuated by the antagonistic SMA springs;

FIGS. 9A-9B represent pictorial views of the MINIR body integrated with the tendon-driven mechanism, irrigation and suction tubes, bi-polar electrocautery probes, and Endoscout® tracking sub-system;

FIGS. 10A-10B represent pictorial views of the base link of the MINIR body encompassing a gear mechanism with the tendons attached thereto;

FIG. 11 is a representation of the intermediate quick-connect mechanism attached between the SMA spring actuators and the base link of the MINIR body;

FIG. 12 shows a quick-connect feature at the base link of the MINIR body;

FIG. 13 shows a quick-connect feature at the actuator side of the system;

FIG. 14 is the representation of the general framework of the operation of the MINIR system;

FIGS. 15A-15D illustrate the principles of the image feedback control of the MINIR; and

FIG. 16 is the flow chart diagram of the overall control process involving the MINIR system of the present invention.

### DESCRIPTION OF THE PREFERRED EMBODIMENTS

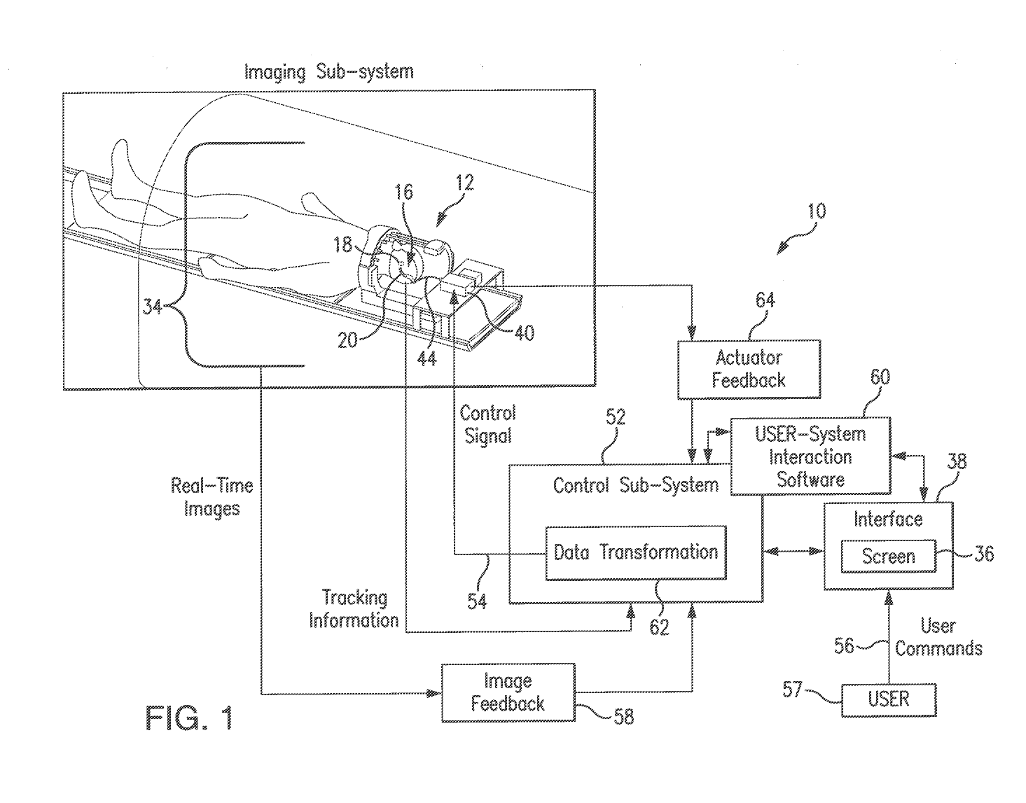

Referring to FIG. 1, a minimally invasive neurosurgical intracranial robot (also referred to herein as MINIR) system 10 includes a robot sub-system 12, which during the surgery is introduced through a narrow surgical corridor to an operative site 16 containing a tumor 18.

The present system will enable the neurosurgeon to remove deep brain intracranial tumors, both neoplastic and non-neoplastic masses, such as blood clots, that are typically hard to reach through a minimally invasive approach, since deep brain tumors are typically located away from the “line-of-sight” of the neurosurgeon. The present system provides the neurosurgeon with means to remove the tumor by teleoperatively navigating the position of the minimally invasive neurosurgical intracranial robot (MINIR) having a number of DOFs (Degrees-of-Freedom) towards the tumor site based on real-time images of the operational site aligned with visual tracking data of the robot, as will be presented in detail in further description.

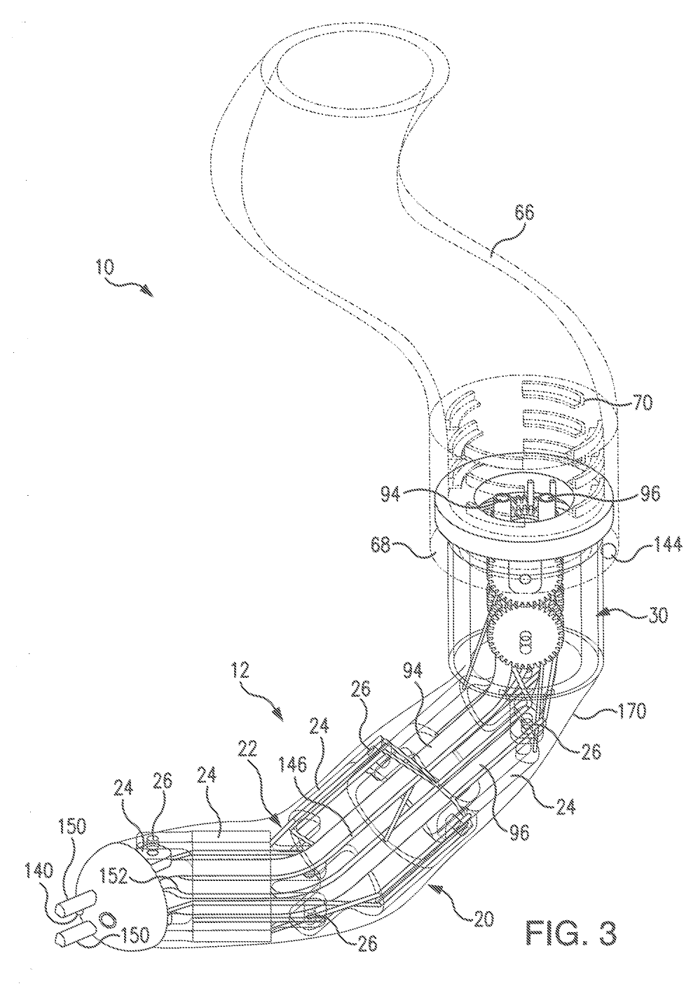

As shown in FIGS. 3 and 5-6, the robot sub-system 12 includes a robot body 20 and a tendon sub-system 22 fully integrated with the robot body 20 and routed through the robot body 20 in a predetermined fashion.

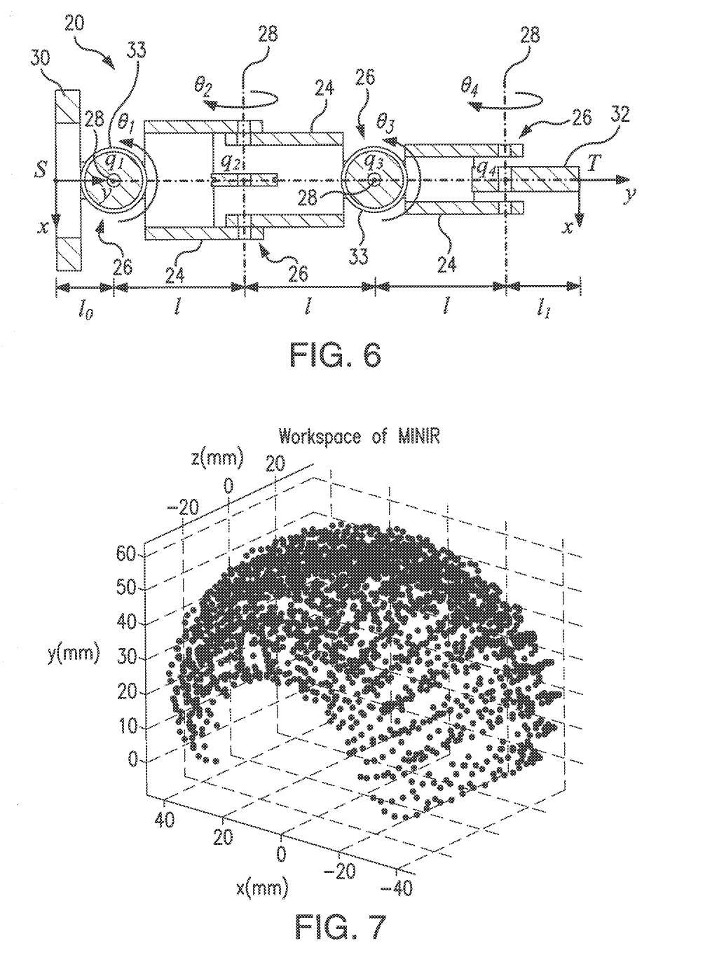

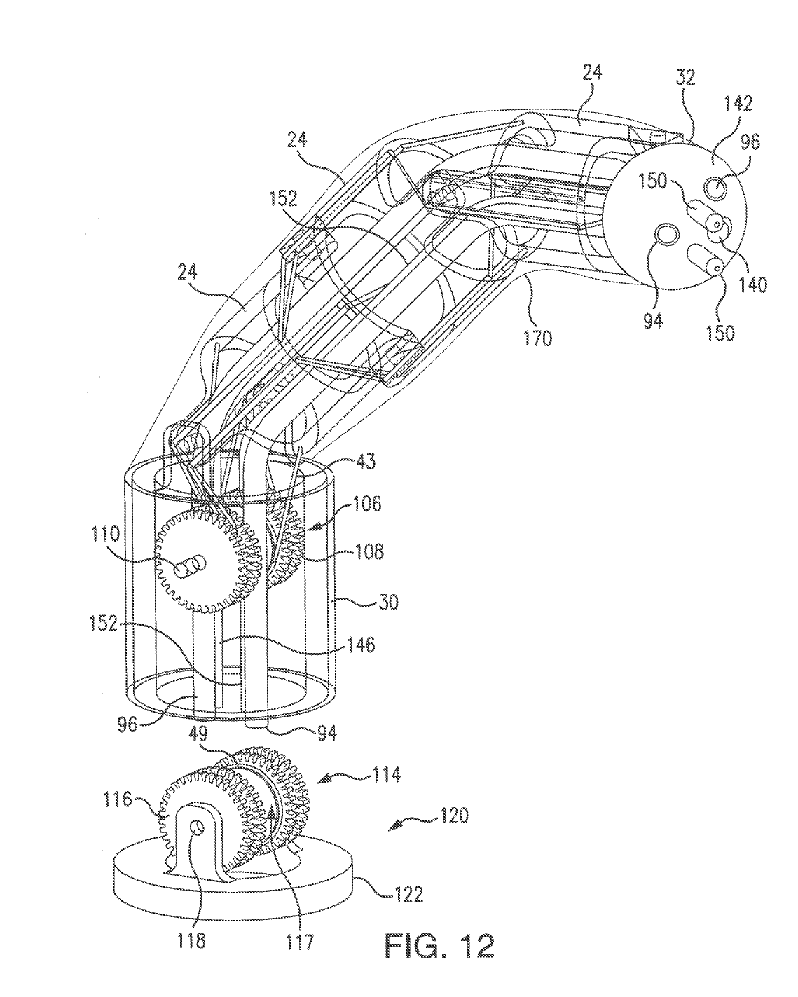

The robot body 20, as best shown in FIGS. 3, 5-6, 8B-8D, 9A-9B, 10A-10B, 12, and 15A-15D, is composed of a plurality of links 24 interconnected through revolute joints 26. Each revolute joint 26 is formed between adjacent links 24 for rotational motion of each link with respect to the other about a corresponding rotational axis 28. As best shown in FIG. 6, each axis 28 extends in substantially orthogonal relationship with an adjacent axis to provide the “out-of-plane” motion for the links.

The number of the links is not limited and may vary depending on the specific surgical operation to be performed. As an example only, but not to limit the scope of protection of the present system to a particular number of the links, in one of the implementations of the MINIR body described herein, the robot body is shown equipped with five links 24. These five links include a base link 30 and a tip link 32, and three intermediate links connected therebetween. In this particular example, the five links are interconnected through four joints 26, although any other number of joints may be formed depending on the number of the links needed for a particular surgical application of the MINIR system in question.

The system 10 operates in conjunction with an imaging system 34 which generates substantially in real-time images of the operative site 16 containing the tumor 18 and provides these images to the screen (or any other display means) 36 on the neurosurgeon's interface 38.

The principles of the present minimally invasive neurosurgical intracranial robot system are fully applicable to a variety of imaging modalities. In order to be used with a particular imaging system, such as, for example, MRI, ultrasound, or CT (computed tomography), etc., which are based on different physical principles, the robot sub-system 12 is to be adapted to be compatible with the particular imaging modality.

As an example, the following description is given for the MINIR system operated in an intraoperative MRI (Magnetic Resonance Imaging) environment. The MINIR sub-system is envisioned to be under the direct control of the neurosurgeon with the targeting information obtained from frequently-updated MRI images which are used to provide virtual visualization of the tumor to the surgeon as the tumor's three dimensional shape changes during the surgery.

For example, when being used with the MRI, all the components of the robot body sub-system 12 will be manufactured with MRI compatible materials to attain minimal or no distortion in Magnetic Resonance Images. In the embodiment compatible with the MRI technology, the links will be made of a plastic, or MRI compatible metals, such as, for example, brass, titanium, etc., and tendon sub-system 22 will contain cables (tendons) routed through sheaths. As an example, the tendons and sheaths can be made from plastic compounds.

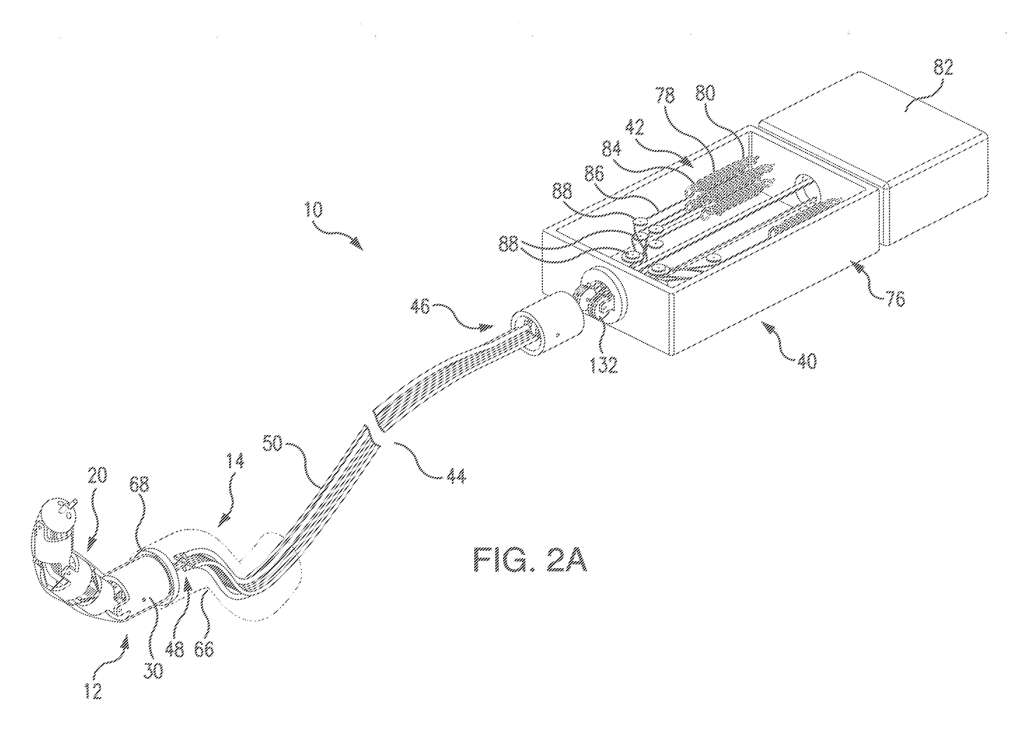

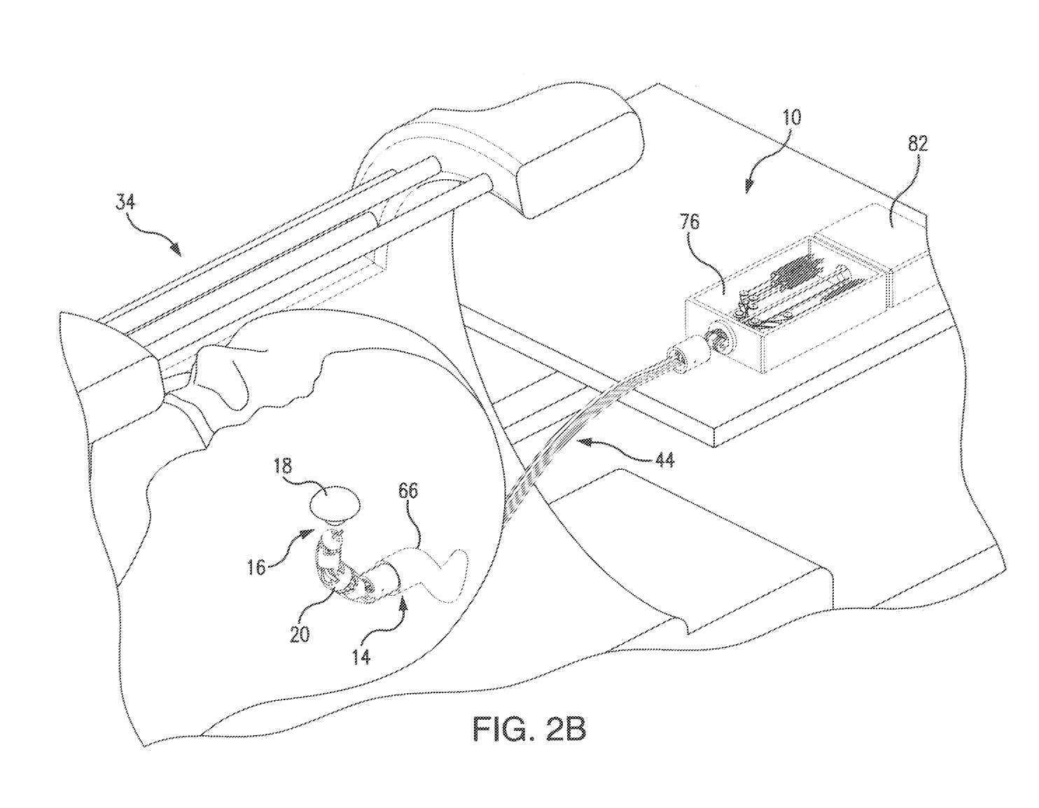

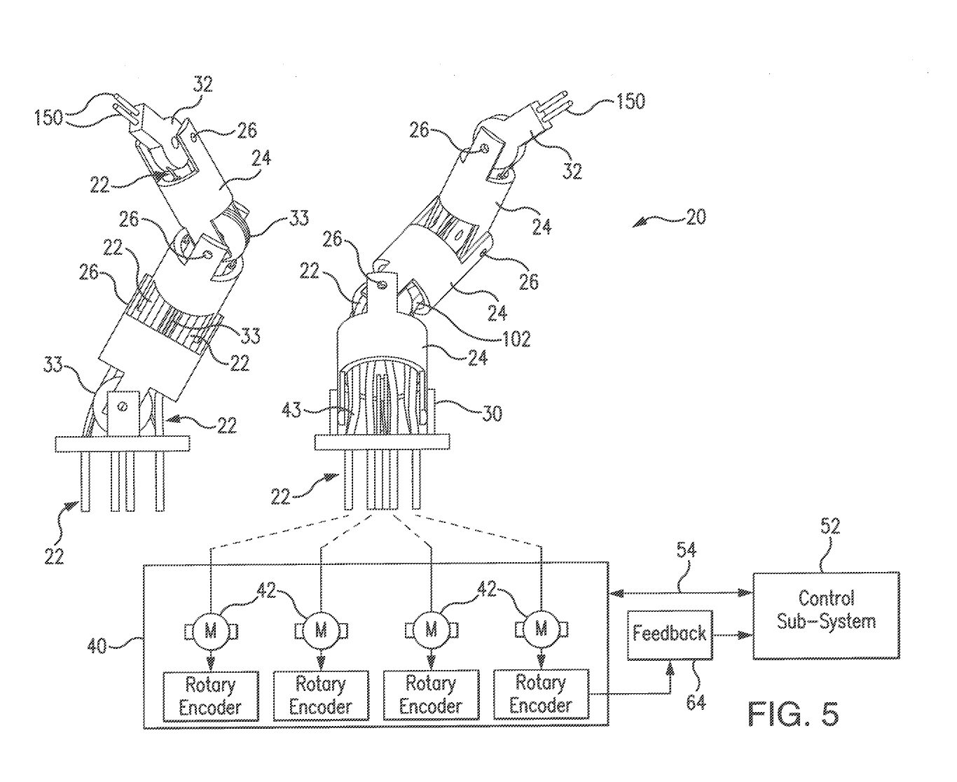

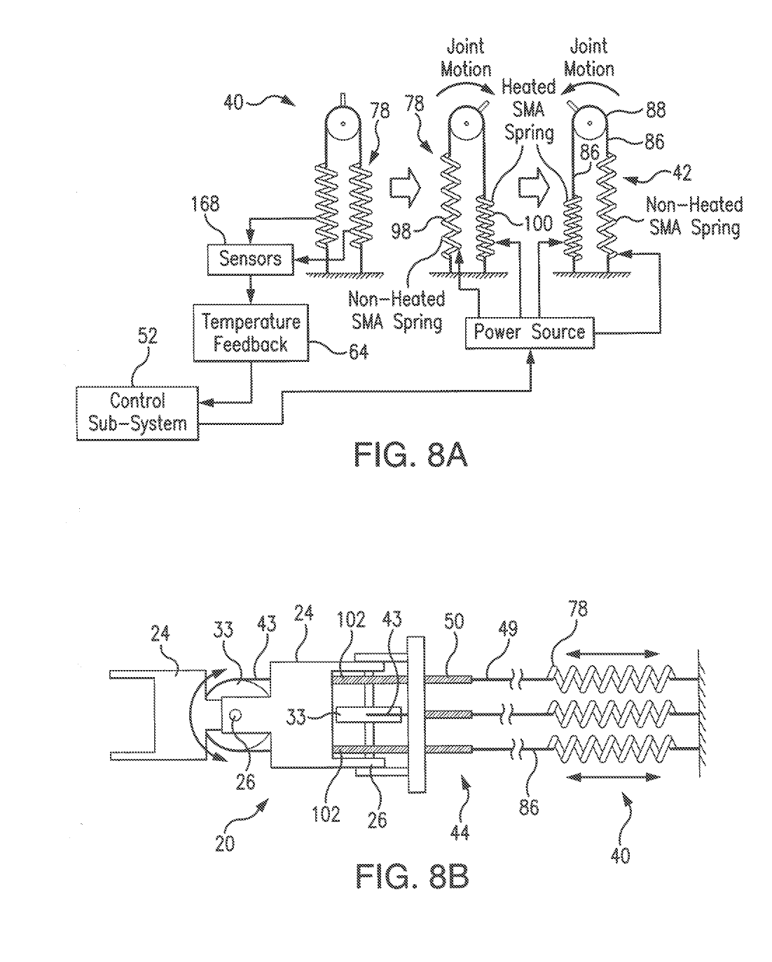

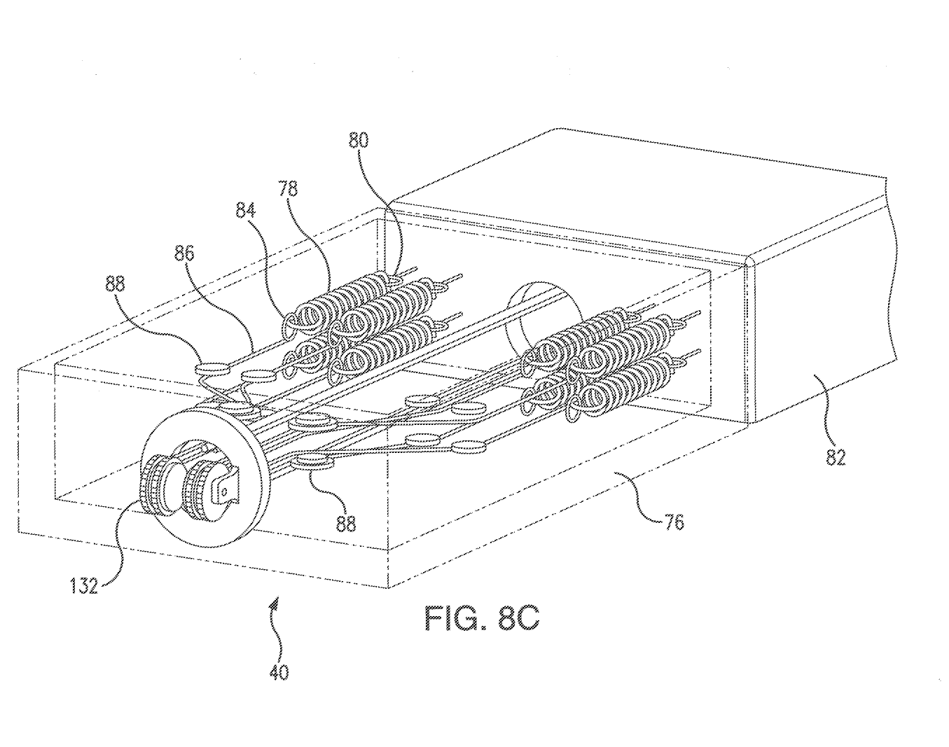

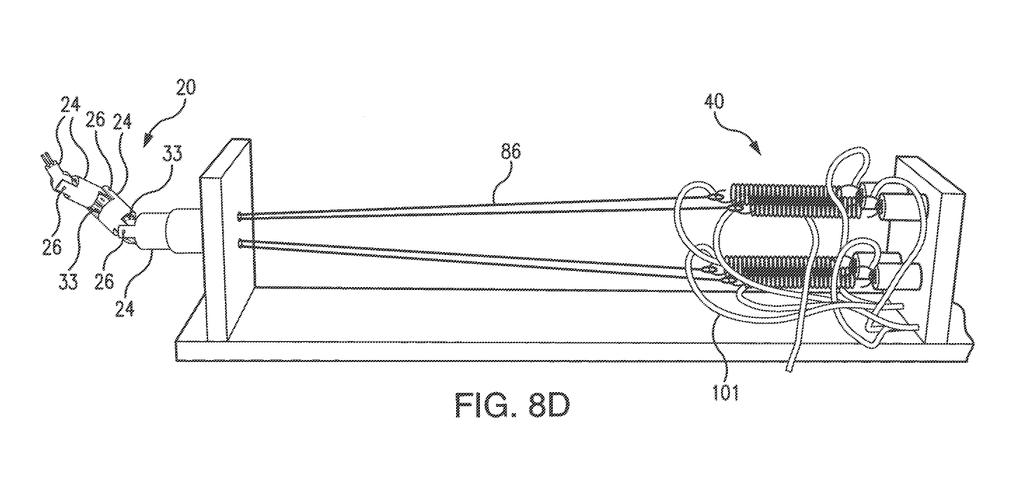

Referring again to FIG. 1, the system 10 includes an actuator sub-system 40. As shown in FIG. 2A, the actuator sub-system 40 includes independent actuating mechanisms 42, the number of which corresponds to the number of revolute joints in the robot sub-system 12. As shown in FIGS. 5 and 8B, each actuating mechanism 42 is operatively coupled to a respective revolute joint 26 in the robot body 20 through a particular tendon 43 in the tendon sub-system to control the revolute motion at each particular joint.

Several embodiments of the actuator sub-system 40 are envisioned herein as will be presented in detail. However, irrespective of its nature, each actuating mechanism 42 independently controls the joint motion of one corresponding joint 26 in a desired direction by controlling the motion of a respective tendon 43 in the tendon sub-system 22.

While MRI provides an extremely restrictive environment when it comes to material, sensors, actuators, etc., choices that can be used, some of these constraints are not present in other imaging modalities, such as, for example, CT and ultrasound. In any case scenario, the actuator sub-system 40 is preferably positioned away from the imaging system to reduce (or completely eliminate) image distortion. In order to reduce (or completely eliminate) the noise and distortion to the images which may be caused by operation of the actuator sub-system 40, the actuator sub-system 40 is positioned in spaced apart relationship with the imaging system 34.

The operative coupling between the actuator sub-system 40 and the tendon sub-system 22 in the present design is provided through an intermediate quick-connect mechanism 44, the proximal end 46 of which is attached to the actuator sub-system 40, while the distal end 48 is attached to the MINIR robot base link 30, as best presented in FIGS. 2A-2B, 11, and 12-13. The intermediate quick-connect mechanism 44 includes cabling 49 passing through plastic sheaths 50.

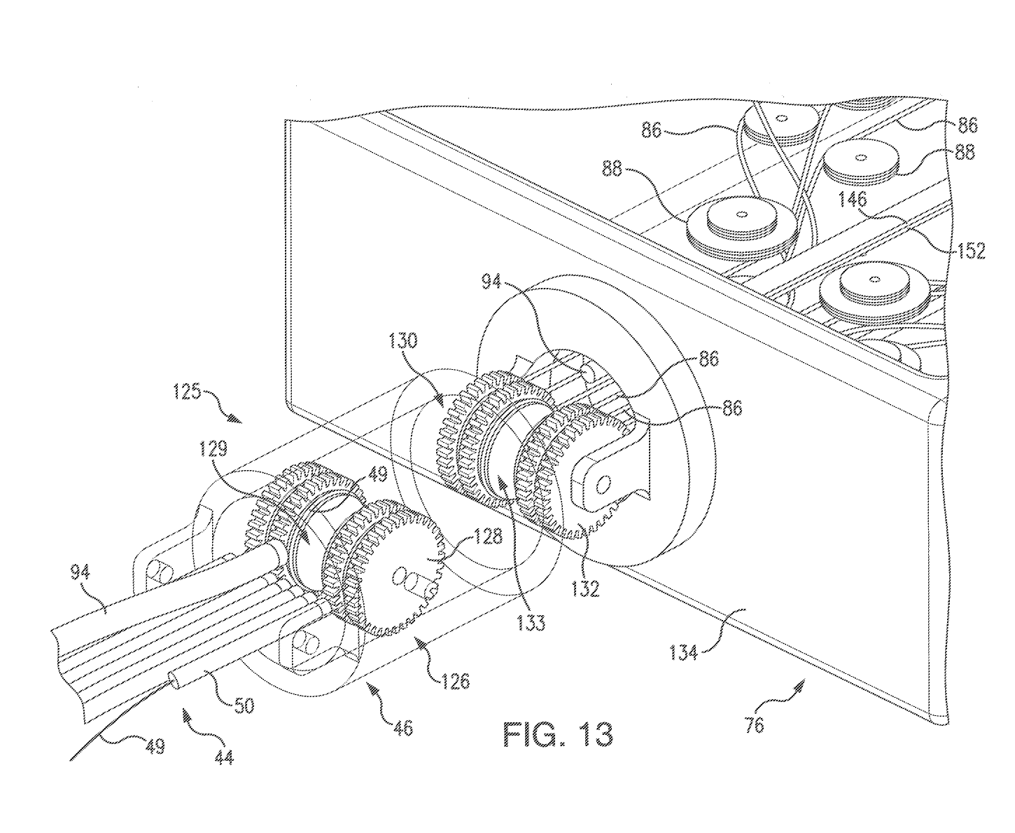

In addition, as shown in FIG. 13, plastic sheaths 50 may be used in the intermediate quick-connect mechanism 44 to route therethrough the wiring for tracking sub-system (for example Endoscout® tracking system), as well as wiring for probes of surgical modalities, which may be used for a particular procedure intended to destroy the tumor tissues, including but not limited to mono- or bi-polar electrocautery, laser and/or radio frequency ablation, ultrasound cavitation, etc. In addition, the intermediate quick-connect mechanism 44 includes tubes 94, 96 routed therethrough for suction and irrigation procedures provided at the robot sub-system 12 to enable the removal of the tissues when needed. For these purposes, the suction and irrigation tubes are entered into contact with the operative site.

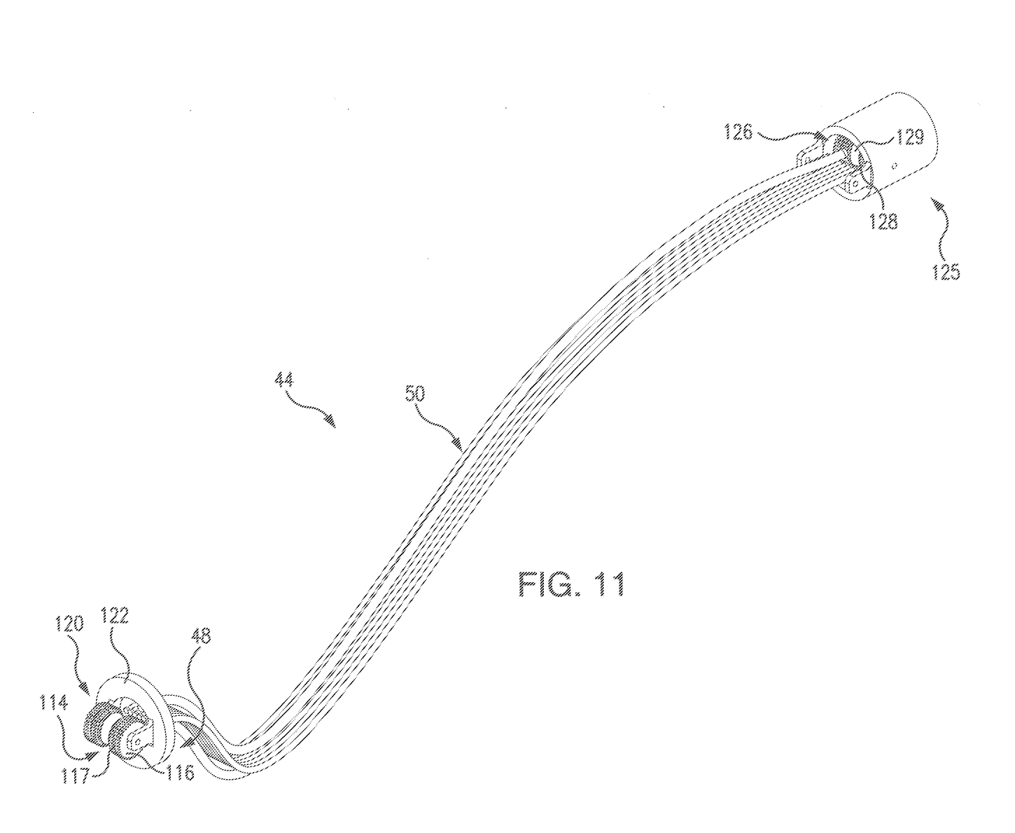

The tendons are pre-tensioned and are maintained in tension during the entire operation. The tendons in the intermediate quick-connect mechanism 44 themselves are not pre-tensioned when they are not connected between the actuator box and the robot body (such as shown in FIG. 11, for example) or not in operation (such as shown in FIGS. 2A and 2B, for example). It is important to note that all the tendons in the system are in pre-tension immediately before the operation of the robot initiated.

Returning to FIG. 1, the system 10 further includes a control sub-system 52 which is operatively coupled between the interface 38 and the actuator sub-system 40. The control sub-system 52 generates control signals 54 responsive to the neurosurgeon's commands 56 entered by the neurosurgeon 57 into the interface 38. The control signals 54 generated by the control sub-system 52 are applied to the actuator sub-system 40, which, responsive to the control signals 54 received thereat, actuates a respective actuating mechanism 42 to control the motion of the tendons 43 in the tendon sub-system 22. This causes rotational motion of the respective links at the revolute joints to steer the robot sub-system 12 towards the tumor 18 at the direction of the neurosurgeon.

The control sub-system 52 calculates the center of rotation at the robot body, i.e., the coordinates of the joint to be affected, and actuates the actuating mechanism 42 corresponding to a joint to control that specific joint independently of others.

The neurosurgeon's instructions to steer the robot body 20 are based on the MRI images received at the screen 36 of the interface 38. In addition, the neurosurgeon is provided with the tracking information acquired by the tracking system integrated with the robot body 20. The tracking information may be in the format of coordinates of the tip (or other part) of the robot.

The tracking system, in conjunction with the generated tracking information, forms an image feedback control sub-system 58 which provides the neurosurgeon with the visual information of the robot body's position, i.e., the response to the commands 56. In other words, based on the tracking information aligned with (or superimposed on) the MRI images at the screen 36 of the interface 38, the neurosurgeon monitors the efficiency of the teleoperative steering of the robot body's in a predetermined manner relative to the tumor in performance of the surgical procedure.

The control sub-system 52 and the interface 38 operate in accordance with User-System Interaction Software 60 which supports the overall control process, as will be further presented. The control sub-system 52 further includes data processor 62 which transforms the neurosurgeon's commands 56 into the control signals 54. The data processor 62, responsive to the commands 56, calculates the center of rotation at the robot body 20, i.e., the coordinates of the revolute joint 26 to be actuated, as well as operational parameters for the actuating mechanisms 42 in the actuator sub-system 40.

As presented in FIGS. 8A-8D, the actuating mechanisms 42 may be implemented as SMA (Shape Memory Alloy) actuators. Alternatively, as presented in FIG. 5, the actuating mechanisms may be implemented with motors.

In the case of the SMA actuators, the data processor 62 calculates a temperature to which the particular SMA actuator is to be heated, and the corresponding electrical current supplied thereto as will be further described.

In the case of employing motors for actuating mechanisms, the data processor 62 will calculate the regime of the motor operation in order to provide a needed motion of particular cables 49 in the intermediate quick-connect mechanism 44, and the motion of the corresponding tendons 43 of the tendon sub-system 22.

Specifically, the following modifications of the actuator design are envisioned in conjunction with the present MINIR system including, but not limited to, the SMA springs actuators, DC motors, or Piezo LEGS rotary motors (manufactured by PiezoMotor, Uppsala, Sweden), with a choice of the actuator sub-system 40 dependent on the choice of the imaging modality.

For example, if DC motors are used, each DC motor may be equipped with a rotary encoder (shown in FIG. 5) and a high gear ratio which is used to give the robot sub-system both fine motion and high output torque. In an alternative embodiment, shown in FIGS. 8A-8D, the actuating mechanisms 42 may be presented by Shape Memory Alloy (SMA) springs.

The number of the actuating mechanisms 42, either motor based or SMA spring based, corresponds to the number of the revolute joints in the robot sub-system to provide an independent control of the tendons 43 and hence the joints 26. The number of degrees of freedom of the system automatically decides the number of the actuating mechanisms 42.

The system 10 also includes an actuator feedback control sub-system 64, shown in FIGS. 1, 5, and 8A.

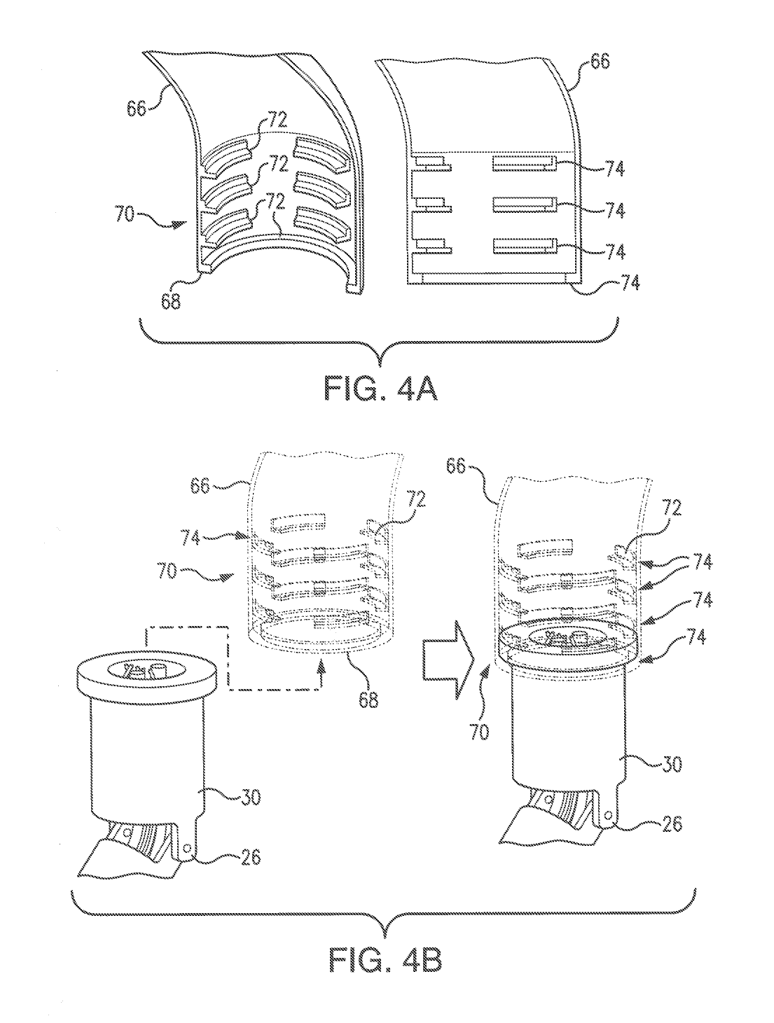

Referring to FIGS. 2A-2B, the MINIR robot sub-system 12 is delivered at the operative site 16 through a flexible cannula 66 which is inserted by the neurosurgeon into the narrow surgical channel. The neurosurgeon advances the MINIR body as much as it is required through the cannula 66 into the operative site 16. As shown in FIGS. 3 and 4A-4B, the cannula 66 is provided with the latching mechanism 70 designed for securing the base link 30 at the distal end 68 of the cannula 66.

The basic component of the latching mechanism 70 may, for example, include semi-rigid rubber tabs 72 provided at the interior wall of the cannula 66 and positioned circumferentially, thus forming several layers 74 at different distances from the edge on the distal end 68 of the cannula 66. The number of the layers 74 of the latching mechanism 70 at the interior wall of the cannula 66 is widely variable.

As an example, four layers 74 are shown in FIGS. 4A-4B. Each layer may include the L-shaped flexible tabs 72. The base link 30 of the robot body 20 is fixed between two layers 74 of the latching mechanism, as shown in FIG. 4B.

During the surgery, the neurosurgeon advances the MINIR body 20 to a position required for the procedure, and the MINIR's base link 30 is latched between the respective layers 74 of the tabs 72. The latching mechanism 70 thus enables the neurosurgeon to control the appropriate amount of the protrusion of the MINIR body through the cannula 66, and hence the depth to which the MINIR body protrudes into the brain.

The exemplary latching mechanism design using the semi-rigid rubber tabs 72 positioned along the inner circumference of the cannula 66, as shown in FIGS. 3 and 4A-4B, requires some effort to deform and hence enable the MINIR's base link 30 to push through and be held in place once it passes through the semi-rigid rubber tabs. For retraction of the MINIR body, a similar level of effort would be required to pull it out and into the cannula once the procedure is completed.

Referring to FIG. 8C, and again to FIGS. 2A-2B, the actuator sub-system may be encased into the actuator box 76 which, in one embodiment, includes SMA antagonistic spring actuators 78 coupled by one end 80 to a hardware routing box 82 which contains the Endoscout®, electrocautery, suction, and irrigation hardware. An opposite end 84 of the SMA springs 78 is coupled through the cables 86 and through intermediate routing pulleys 88 to the gears 132 extending outside of the actuator box 76. The cables 86 routed through the intermediate routing pulleys 88 are in tension during the operation.

In order to be MRI compatible, all components of the robot sub-system 12 may be formed of a plastic composition. Also, links and pulleys can be made from MRI compatible metals including but not limited to brass, titanium, etc. Each revolute joint 26 has an independent single degree of freedom controlled by the actuating mechanism 42 in the motion range for each joint of approximately +/−90 degrees.

Referring to FIG. 8A, each joint of the MINIR is connected to a pair of antagonistic SMA spring actuators 98-100 which allow control of the joint motion in both directions independently. FIG. 8B shows the first link of MINIR connected to a pair of SMA spring actuators through a tendon-sheath (49/50) mechanism, where each SMA spring 78 is actuated independently.

When one of the spring 98 or 100 is heated by applying electric current thereto, the tension in the heated SMA spring increases as opposed to another non-heated SMA spring, thereby causing the joint motion in the direction of the heated SMA spring.

As shown in FIG. 8C, the antagonistic SMA springs 78 corresponding to the number of revolute joints in the robot body, are positioned in the actuator box 76. For the particular example described herein, where the robot body includes four revolute joints, four pairs of antagonistic springs 98-100, shown in FIG. 8C, are positioned in the actuator box 76. By applying electric current to a specific one of the springs in the SMA spring actuator 78, the spring cables 86 will rotate (through the corresponding pulley 88) the gears 132 (shown in FIG. 13) in the direction corresponding to the heated/non-heated antagonistic springs 100/98.

The rotational motion of the gears 132 will be transferred through the intermediate quick-connect mechanism 44, through the system of gears and a respective tendon 43, to a respective joint of the robot body. As shown in FIG. 8B, the SMA springs 78 are connected through the tendons (cables) 86 (pulleys 88 are not shown in this particular schematic) to the robot. This in turn is operatively coupled to the tendons 43 routed through the robot body 20 and the pulleys 33 provided at the joints 26 to steer the robot as needed.

### Motion of MINIR

MINIR, as shown in FIG. 5, is an open-chain manipulator where each pair of links 24 is connected by a revolute joint 26. Assuming that the reference configuration (θ₁=θ₂=θ₃=θ₄=0) of MINIR is fully extended, as shown in FIG. 6, the forward kinematics can be computed. The workspace of MINIR can be computed by some choice of joint angles θᵢ (i=1, . . . , 4). The workspace of MINIR is shown in FIG. 7 where the origin (0, 0, 0) is the center of the base link of the robot body.

### Actuation Design

SMA is MRI compatible, however, the electric current used to actuate it may cause noise and image distortion in the MR images. To improve the MR image quality and minimize the disturbance caused by the electric current, in the present system, the SMA actuators are moved away from the imaging region. The tendon-sheath mechanism 44 is used to transmit the actuation force of the SMA spring actuators 78 to each joint of the MINIR. Experimental results showed that the SNR (signal-to-noise ratio) of the MR images only dropped by about 1.2% after actuation. This design not only attains the improved MR image quality but can also prevent damage to the surrounding brain tissue which may be caused by the heat generated by electric current applied to the SMA springs.

The SMA spring actuators with tendon-sheath mechanism has proved to be an effective actuation technique for the MINIR. To use SMA springs as actuators, their behavior has been modeled and characterized.

The one-dimensional shear stress and shear strain relation of SMAs can be expressed as (C. Liang and C. A. Rogers, “Design of shape memory alloy springs with applications in vibration control,” J. Intel. Mat. Syst. Str., vol. 8, no. 4, pp. 314-322, 1997):

$$τ - {τ}_{0} = G ( γ - {γ}_{0} ) + \frac{Ω}{\sqrt{3}} ( ξ - {ξ}_{0} ) + \frac{Θ}{\sqrt{3}} ( T - {T}_{0} ) \qquad ( Eq . 1 )$$

where τ, γ, ξ, T, G, Ω, and Θ are the shear stress, shear strain, martensite volume fraction, temperature, shear modulus, phase transformation coefficient and thermal expansion coefficient of the SMA spring, respectively.

τ₀, γ₀, ξ₀, T₀ are the initial conditions of the SMA spring. Since the thermal expansion effect is much less than the phase transformation effect, the thermal expansion term may be neglected.

The phase transformation coefficient is a material constant and may be defined as (C. Liang and C. A. Rogers, “Design of shape memory alloy springs with applications in vibration control,” J. Intel. Mat. Syst. Str., vol. 8, no. 4, pp. 314-322, 1997):

Ω=−\sqrt{3}Gγ_{L}, where γ_{L} is the maximum recoverable shear strain of SMA. Therefore, the above (Eq. 1) may be simplified as:

τ−τ₀=G(γ−γ₀)−Gγ_{L}(ξ−ξ₀) (Eq. 2)

For a helical spring, the shear stress, τ, and shear strain, γ, may be correlated with the spring force, F, and spring displacement, δ, respectively. The relations may be written as (J. E. Shigley and C. R. Mischke, Mechanical Engineering Design. McGraw Hill, 2001):

$$τ = {k}_{s} \frac{FD}{π {r}^{3}} δ = \frac{π {D}^{2} N}{2 r} γ \qquad ( Eq . 3 )$$

where D is the diameter of the spring, N is the total number of active coils in the spring, r is the radius of the spring wire, and kₛ is the Wahl correction factor.

The shear modulus, G, may be computed using:

$$G = \frac{E}{2 ( 1 + v )} \qquad ( Eq . 4 )$$

where v is the Poisson's ratio and E is the Young's modulus of the SMA spring which is given by (K. Tanaka, “A thermomechanical sketch of shape memory effect: One dimensional tensile behavior,” Res. Mechanica, vol. 18, no. 3, pp. 251-263, 1986):

E(ξ)=E_{A}+(E_{M}−E_{A})ξ (Eq. 5)

where E_{M} and E_{A} are the Young's modulus of the martensite phase and austenite phase of the SMA spring, respectively.

Substituting the above (Eq. 3) and (Eq. 4) into (Eq. 2) yields:

C₁(F−F₀)=C₂E(ξ)(δ−δ₀)−C₂E(ξ)δ_{L}(ξ−ξ₀) (Eq. 6)

where F₀ and δ₀ are the initial force and initial displacement of the SMA spring, respectively.

C₁ and C₂ are constants and are defined as:

$${C}_{1} = \frac{D {k}_{s}}{π {r}^{3}} {C}_{2} = \frac{r}{π {D}^{2} N ( 1 + v )} \qquad ( Eq . 7 )$$

The above (Eq. 6) may be used to describe the force-displacement relation of a SMA spring.

Note that if the temperature in a SMA spring stays constant, no phase transformation can occur (ξ−ξ₀=0). Therefore, (Eq. 6) can be written as:

$$F - {F}_{0} = \frac{{C}_{2}}{{C}_{1}} E ( ξ ) ( δ - {δ}_{0} ) \qquad ( Eq . 8 )$$

The above (Eq. 8) implies that the SMA spring can be used as a regular helical spring when the temperature in the SMA spring stays constant. The spring constant, k, can be expressed as:

$$k = \frac{{C}_{2}}{{C}_{1}} E ( ξ ) \qquad ( Eq . 9 )$$

### Antagonistic SMA Spring Analysis

Two SMA springs (with original length L) as shown in FIG. 8A, were analyzed. One of the springs was pre-stretched by an amount of δ₀ and then recovered by δᵣ when actuated. Since the non-actuated SMA spring may be used as a regular helical spring, the recovery displacement, δᵣ, can be expressed as:

$${δ}_{r} = \frac{F - {F}_{0}}{k} \qquad ( Eq . 10 )$$

Substituting (Eq. 10) into (Eq. 6), there is obtained:

$$F - {F}_{0} = \frac{{C}_{2}}{{C}_{1}} E ( ξ ) ( - \frac{F - {F}_{0}}{k} ) - \frac{{C}_{2}}{{C}_{1}} E ( ξ ) {δ}_{L} ( ξ - 1 ) \qquad ( Eq . 11 )$$

The martensite volume fraction, ξ, can be derived based on transformation kinetics. For a heating transformation (martensite to austenite), it can be expressed as (C. Liang and C. A. Rogers, “Design of shape memory alloy springs with applications in vibration control,” J. Intel. Mat. Syst. Str., vol. 8, no. 4, pp 314-322, 1997):

ξ_{M→A}=½{ cos [a_{A}(T−Aₛ)+\sqrt{3b}_{A}τ]+1} (Eq. 12)

where a_{A} and b_{A} are material constant and Aₛ is the transformation temperature of SMA.

Since ξ is a cosine function of τ, (Eq. 11) can be solved numerically when T is known using Newton-Raphson's method, which is given by:

$$\begin{matrix} {F}_{new} = {F}_{old} - \frac{f ( {F}_{old} )}{{f}^{′} ( {F}_{old} )} \\ where & ( Eq . 13 ) \\ f ( F ) = F - {F}_{0} + \frac{{C}_{2}}{{C}_{1}} E ( ξ ) ( \frac{F - {F}_{0}}{k} ) + \frac{{C}_{2}}{{C}_{1}} E ( ξ ) {δ}_{L} ( ξ - 1 ) & ( Eq . 14 ) \\ {f}^{′} ( F ) = 1 + \frac{{C}_{2}}{{C}_{1}} \frac{F - {F}_{0}}{k} \frac{\partial E ( ξ )}{\partial F} + \frac{{C}_{2}}{{C}_{1}} E ( ξ ) \frac{1}{k} + \frac{{C}_{2}}{{C}_{1}} \frac{\partial E ( ξ )}{\partial F} {δ}_{L} ( ξ - 1 ) + \frac{{C}_{2}}{{C}_{1}} E ( ξ ) {δ}_{L} \frac{\partial ξ}{\partial F} & ( Eq . 15 )\end{matrix}$$

In summary, recovery force, F, of the SMA spring actuator can be predicted using (Eq. 11) once the temperature, T, is known.

(Eq. 10) can be used to calculate the recovery displacement of the SMA spring. Thus, the joint motion can be controlled, and the output force of MINIR can be estimated using an actuator feedback control 64 (shown in FIG. 1), which may be implemented, in one embodiment, as a temperature feedback 64 shown in FIG. 8A.

Shown in FIG. 8D is an example of controlling the MINIR prototype where four SMA springs were used to actuate two joints of the robot body. The two joints may be actuated to move in orthogonal directions, and each joint can be controlled independently and precisely by using either image feedback control and/or the temperature feedback control.

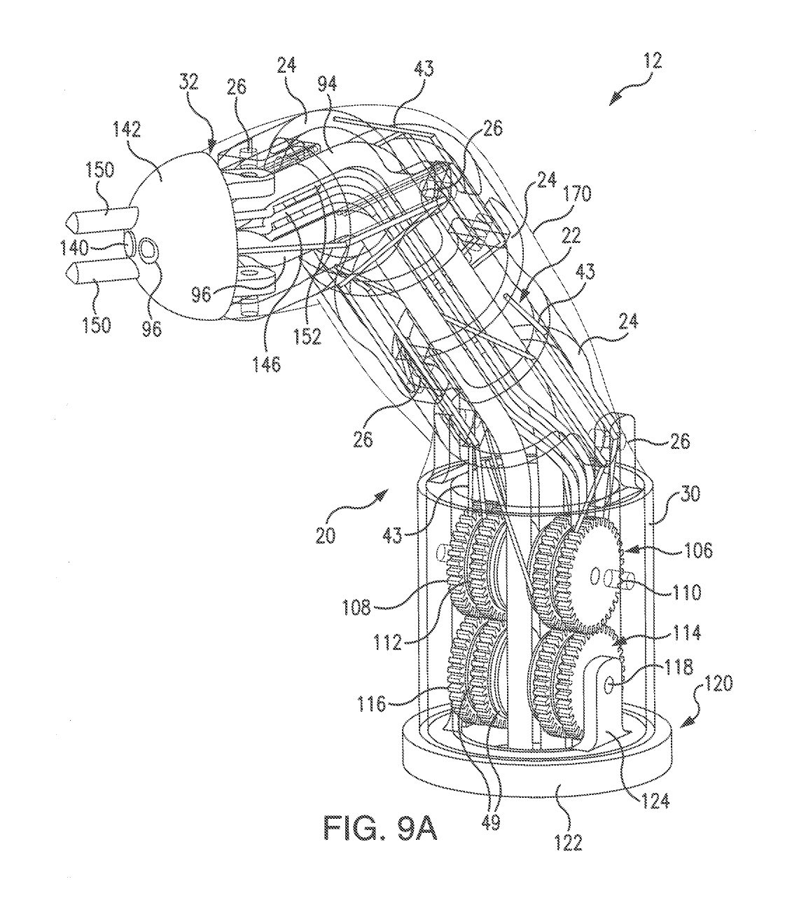

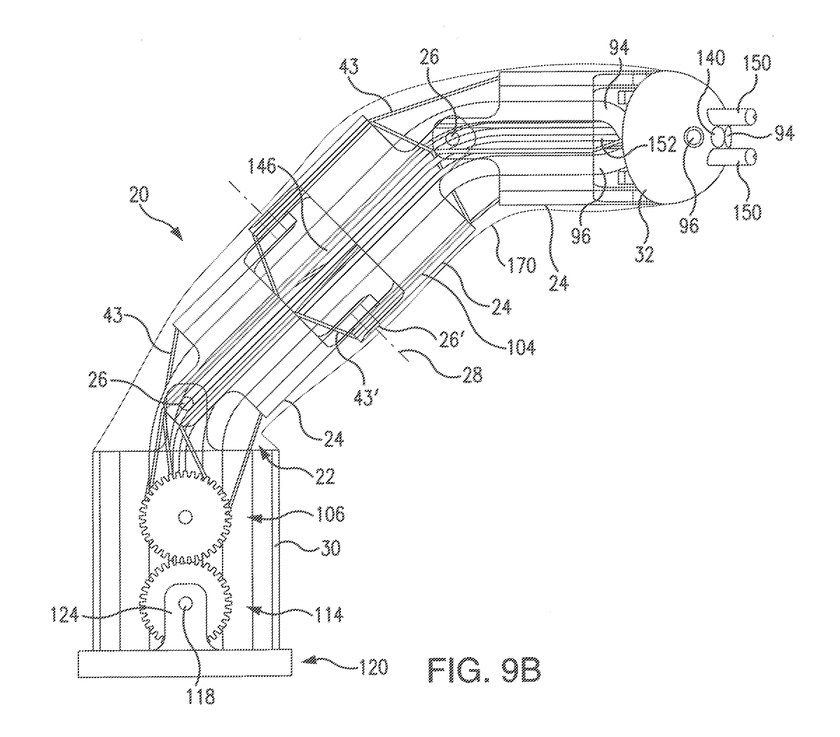

Referring to FIGS. 9A-9B, showing in detail various views of the robot sub-system 12 of the present invention, and returning to FIG. 5, the robot body 20 includes links 24 composed of the base link 30, tip link 32 and intermediate links positioned therebetween which are interconnected by the revolute joints 26.

The tendon sub-system 22 is integrated in the robot body 20. The tendon sub-system 22 includes tendons 43 which are routed through the channels 104 formed within the walls of the links 24. The tendons 43 transition between the links in a manner providing a minimal (ideally zero) torque applied to the joints which are not supposed to be moving.

Each tendon 43 of the tendon sub-system 22 is routed within a corresponding sheath 102 (shown in FIG. 5) as close to the axes 28 of the joints 26 as possible to prevent rotation at a joint whose motion is not desired.

For example, as shown in FIG. 9B, the portion of the sheath with the tendon 43′ inside is routed close to the axis 28 of the joint 26′ so that when it is desired to move the next joint (i.e., adjacent to the joint 26′), there is no (or negligible) motion of the joint 26′. The routing of the tendons (along with their respective sheaths) may be chosen empirically or based on calculated functions. The correct channeling of the tendons through the walls of the links may also be chosen based on the available space inside the robot body 20.

The pulleys 33 may be positioned between adjacent links 24 at the joints 26, as shown in FIGS. 5, 6, 8B and 8D, to facilitate correct routing of tendons/sheaths, as well as needed wiring and irrigation (and suction) tubes through the hollow robot body 20.

Referring to FIGS. 9A-9B, 10A-10B, 11, and 12-13, the subject system 10 is equipped with a system of gear/pulley pairs to control the motion of the tendons 43 for controllable steering of the robot body 20.

As shown in FIGS. 9A-9B, 10A-10B, and 12, a first set 106 of gears 108 (the number of which corresponds to the number of revolute joints 26 in the robot body 20) is positioned within the base link 30 at the shaft 110 secured to the walls of the base link 30.

Each of the gears 108 is integrated with a pulley 112, thus forming a gear/pulley pair. The gear/pulley pair may be either in a form of a single fused member or in the form of a rigidly connected unit where the tendon (within the sheath) is routed around the pulley portion 112 of each gear 108. The tendons may be initially pre-tensioned. In the process, the tendon on each pulley independently controls the joint motion for one joint in one particular direction in correspondence with the action of a particular actuating mechanism 42. The motion of each joint 26 in either direction is achieved through control of each tendon 43 by the action of the respective actuating mechanism.

In order to provide the transformation of the action of the actuating mechanisms 42 into controlled motion of the tendons 43 in the tendon sub-system 22 integrated with the robot body 20, a second set 114 of gear/pulley pairs 116/117 positioned on the shaft 118, can be attached to the base link 30, as shown in FIGS. 9A-9B, 10A-10B, 11, and 12, through a quick-connect mechanism 120.

The gearing in the present system is provided for the motion and torque amplification as appropriate to the function of the system.

The quick-connect mechanism 120 is positioned at the distal end 48 of the intermediate quick-connect mechanism 44 (shown in FIGS. 2A, 2B, 8B and 11), and includes a circular base 122 which is provided with tabs 124 supporting a shaft 118 with gears/pulleys 116.

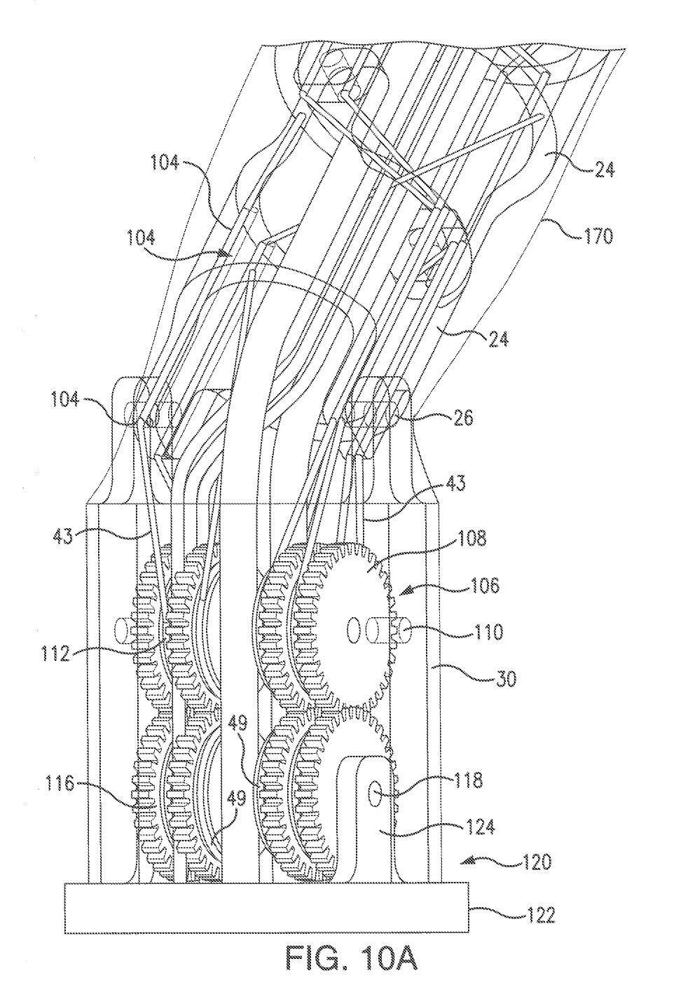

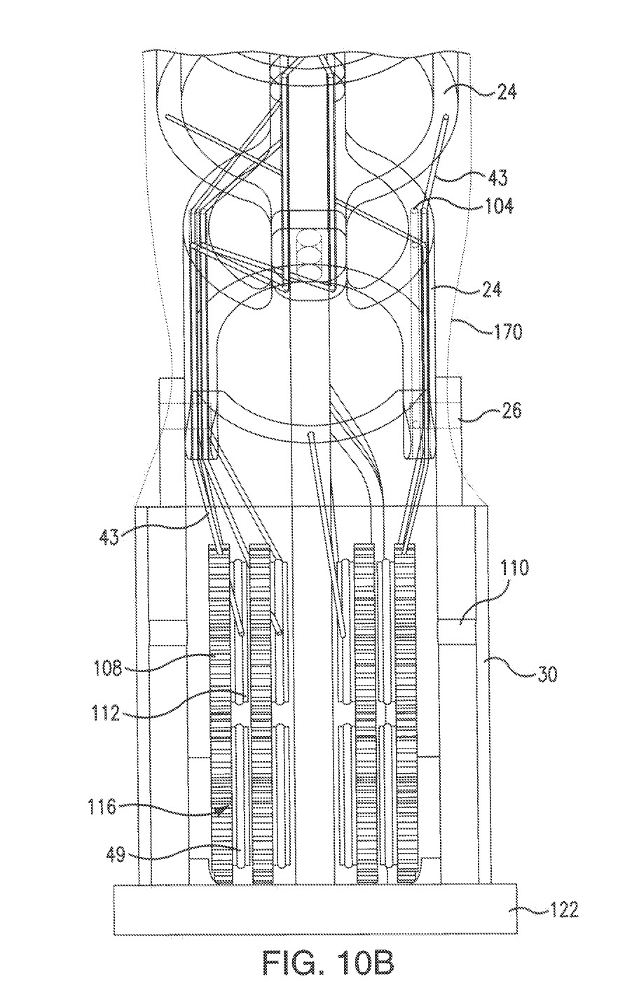

As shown in FIGS. 9A-9B and 10A-10B, the circular base 122 may be removably attached to the base link 30 so that the gear/pulleys units 116 of the second set 114 are engaged with respective gear/pulleys pairs 108/112 of the first set 106 in order to transfer the motion of the gear/pulleys 116/117 into motion of the gear/pulleys 108/112. This in turn, is transformed into the motion of the tendons for steering of the robot body 20. Due to plasticity of the walls of the base link 30 and positioning of the tabs 124 on the circular base 122 for intimately engagement with the inner walls of the base link 30, a quick coupling therebetween may be attained when the gear/pulleys 116 are inserted into the base link 30 and the walls of the base link 30 snuggly embrace the tabs 124, thus securing the quick-connect feature 120 to the base link 30. In alternative embodiment, the base link 30 may be provided with a latching mechanism similar to that provided at the distal end of the cannula (as shown in FIGS. 4A-4B), arranged by two layers of tabs formed at the internal wall of the base link to latch the circular base 122 therebetween.

At the proximal end 46 of the intermediate quick-connect mechanism 44 (as shown in FIGS. 11 and 13), there is a quick-connect mechanism 125 consisting of a third set 126 having gear/pulleys pairs 128/129. The third set 126 is connected to the fourth set 130 of the gear/pulleys units 132/133 positioned at the wall 134 of the actuator box 76.

As discussed in previous paragraphs, the actuation of a corresponding SMA spring is transformed (through the cables 86 and the pulleys 88) into rotation of a respective gear/pulley units 132 which, in turn, is transformed into the controlled motion of a respective cable 49 in the intermediate quick-connect mechanism 44 resulting in rotation of a gear/pulley unit 116 in the second set 114 which correspondingly rotates the gear/pulley 108/112 in the first set 106. Such rotation of the gear/pulleys 108/112 sequentially results in the control of the motion of a corresponding tendon 43 routed through the robot body 20, thereby actuating a respective joint and causing rotational motion of one link with respect to the other in correspondence with the control signal 54 responsive to the neurosurgeon's command 56.

As seen in FIGS. 3, 5, 6, 9A-9B, 10A-10B, and 12, the robot body 20 is a hollow body which permits routing of the suction tube 94, irrigation tube 96, as well as the wiring for probes and tracking system inside the robot body 20 thereby further promoting minimization of the robot dimensions.

As presented in FIGS. 3, and 9A-9B, the robot sub-system 12 is integrated with the tracking system which may be one of a variety of tracking systems compatible with imaging technology used for medical purposes. For example, the present system may be equipped with the Endoscout® tracking system (manufactured by Robin Medical Inc.). The Endoscout® tacking system is a system for MRI guided interventions which enables tracking of the location and orientation of miniature sensors during the MRI scan. The tracking is based on the native gradient fields of the MRI scanner.

In the present MINIR robot, one Endoscout® sensor 140 is positioned at the hemispherical tip member 142, while a second Endoscout® sensor 144 is attached at the distal end 68 of the flexible cannula 66. The wiring 146 for the Endoscout® is routed inside the hollow body 20 of the robot from the sensors 140 and 144 along the intermediate quick-connect mechanism 44 to the hardware routing box 82. In one embodiment, the wiring 146 for Endoscout® system passes through the actuator box 76 for compactness of the system.

The hemispherical tip member 142 secured at the tip link 32 carries end-effector (probes) 150 to perform surgical procedures in a number of treatment modalities which may be used for a particular procedure to destroy tissues of the tumor including but not limited to bi-polar electrocautery, laser and/or radio frequency ablation, ultrasonic cavitation, monopolar electrocautery, etc. The wiring 152 for these treatment modalities extends inside the hollow robot body 20 from the probes 150 at the hemispherical member 142 to the hardware routing box 82. The routing of the wiring 152 may preferably be carried out along the intermediate quick-connect mechanism 44 and through the actuator box 76 for ergonomical benefits as well as compactness of the system 10.

Further attached to the hemispherical member 142 at the tip link 32 is the end of the irrigation tube 96 and suction tube 94 which are routed inside the hollow robot body 20 towards the hardware routing box 82 preferably along the intermediate quick-connect mechanism 44 and the actuator box 76. The suction and irrigation channels within the structure enable the treatment modality approach for removal of the tissue. The suction tube 94 is attached to a pump (not shown) at the end opposite to the end extending into the intracranial operative site to controllably remove tumor tissues. The irrigation tube 96 is connected to a liquid reservoir (not shown) to supply the irrigation liquid to the operative site when needed to clear blood and tissue debris.

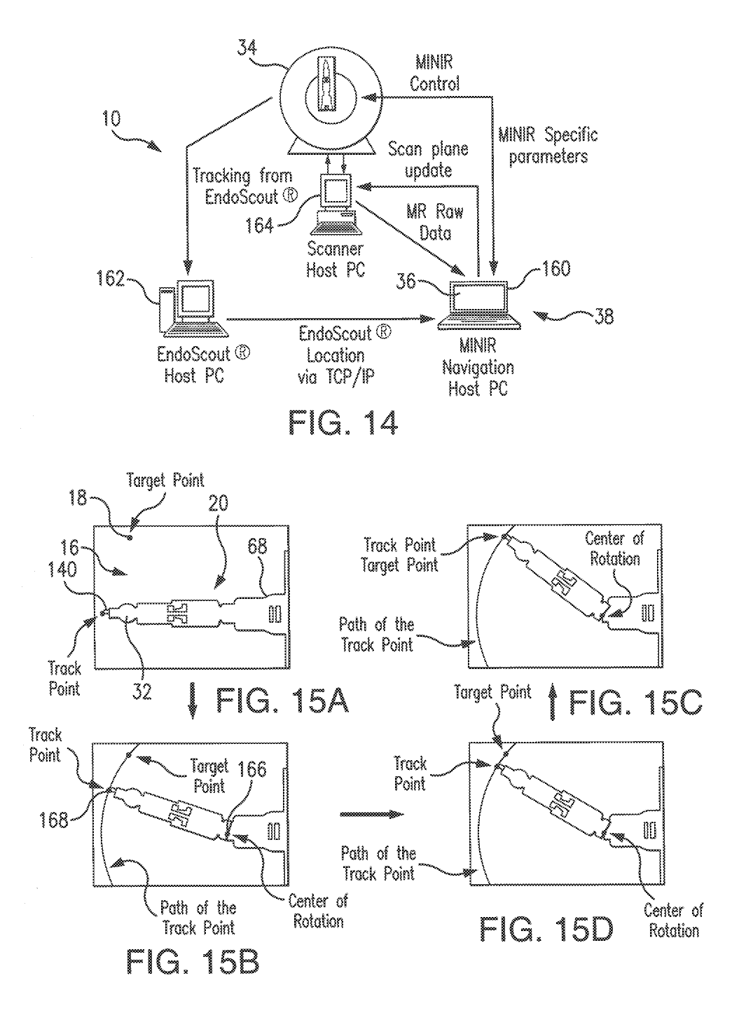

Referring again to FIG. 1, and further to FIG. 14, the user interface 38 may be integrated into the MINIR Navigation Host PC (also referred to as MNHPC) 160. The Endoscout® sensors 140 and 144 located at the tip 142 of the robot body and at the distal end 68 of the cannula 66, respectively (as best shown in FIGS. 3, 9A-9B, and 12), transmit the tracking information corresponding to the position of the robot body to the Endoscout® Host PC 162, which in turn communicates the acquired information to the MNHPC 160. The MNHPC 160 converts the coordinates of the sensors 140, 144 to the MR (Magnet Resonance) coordinates, i.e., aligns the tracking visual with the MR images.

The neurosurgeon visualizes the MR images presented on the screen 36 along with the coordinates of the robot tip, and navigates the robot tip in the desired direction through entering commands 56 into the MNHPC 160.

Accordingly, the commands 56 are transformed into corresponding control signals 54, which are applied to the robot (through the actuating mechanisms). Responsive to the control signals 54, the robot changes its position and/or configuration, and the Endoscout® sensors 140, 144 transform information corresponding to the changes in the position/configuration to the Endoscout® host PC 162, which, in turn, communicates with the MNHPC 160. This process takes place whenever the MINIR body is moved.

The images on the MNHPC's screen 36 may be updated in real-time for the neurosurgeon to analyze the situation and to further follow up with instructions/commands. In order to accomplish this, responsive to the new navigation instructions entered by the physician in the interface 38, the MNHPC 160 generates new coordinates (based on the neurosurgeon's commands for the MINIR) to the Scanner Host PC 164 which, in turn, obtains images corresponding to the new positions of the robot and displays them in real-time on the MNHPC thus providing the feedback routine.

The neurosurgeon may view images just in front of the tip of the MINIR, a coronal view centered around the midpoint of this image, and a sagittal view also centered around the center of this image. In this manner, the neurosurgeon is always able to see the intracranial operation site in front of the MINIR in all three orthogonal views.

The neurosurgeon may choose to terminate navigation, and obtain high-resolution diagnostic quality images which may further help in assessing the margins of the tumor and to make the decision to terminate or to continue further electrocauterization or any other tissue liquefaction modality.

Referring again to FIGS. 1, 5, and 8A, two feedback control sub-systems are used in the present system, including image feedback control 58, and an actuator feedback control 64 which depends on the type of the actuator sub-system. The image feedback control 58 may sometimes fail, due to the noise in the images or due to missing the track point. Since safety is the most important factor for the surgical robot, a backup controller (feedback) 64 is implemented for the MINIR. The actuator feedback control unit 64 may be implemented as a temperature feedback control if the actuator sub-system 40 is built with SMA spring mechanism (as shown in FIG. 8A). Alternatively, if the actuator sub-system 40 is built with motors, as shown in FIG. 5, the actuator feedback control unit 64 may monitor position of the motors (acquired by appropriate sensors, such as for example, rotary encoders) corresponding to the configuration/position of the MINIR, and feed this information to the control sub-system 52.

In the case of the SMA actuators (in addition to the navigation and high-resolution visualization), the MNHPC 160 records temperature from appropriate sensors 168 (schematically shown in FIG. 8A) such as, for example, thermocouples/RTD sensors. The data are made available on demand. The backup control unit 64 monitors the temperature in each SMA spring and uses the temperature feedback as a backup control strategy. This is done primarily because there may be times when the imaging plane of the MRI may not align (due to delays in the repositioning of the operation site slices to be imaged) with the configuration of the robot. In those instances it is desired to enhance the images. For this purpose, the information from the SMA springs is used to determine the joint angle resulting in a needed robot configuration. While the image guided feedback control primarily runs in the foreground, the temperature data are collected from the thermocouples connected to the SMA springs in real-time and stored during robot operation. Alternatively, the back-up control unit 64 monitors the readings of the motors' rotary encoders (as shown in FIG. 5) which are stored during the robot operation and used on demand.

The present system is adaptable to a number of imaging techniques, including MRI, CT, ultrasound, etc. As an example, (not to limit the scope of the present invention to any particular imaging modality) the MINIR system described herein, is specifically suited for MRI pulse sequence to enable communication with the MINIR for real-time imaging manipulations. The pulse sequences used are envisioned, for example, as standard rapid imaging sequences, which are commonly provided by manufacturers of the MR equipment.

Referring again to FIG. 14, the raw data from the imaging system 34 (for example, the MRI equipment) are exported from the Scanner Host PC 164 through the TCP/IP protocol and reconstructed within the MNHPC 160. The MR images are displayed on the MNHPC 160 in real-time to aid surgical navigation. The MNHPC 160 is able to switch from tracking mode to high resolution mode upon the neurosurgeon request, or such switching can be accomplished automatically. Both high resolution and tracking mode imaging techniques are used in this system. Switching from navigation mode to high resolution mode may occur through user interface available on the MNHPC 160.

A majority of the manipulations may be carried out to view the images in the tracking mode (which is in real-time mode of operation) to learn the position of the MINIR, or to obtain high-resolution images with desired contrast to assess whether to stop, change direction, or continue with the tissue liquefaction.

In one envisioned embodiment, the imaging manipulation may require the interface 38 to have a touch pad display or a joy stick to manipulate the direction in which the MINIR should move. The software on the MNHPC 160 may be in a basic (or default) version, or may be flexible enough to accommodate the surgical practices of each neurosurgeon and their workflow. This may be provided through software modules incorporated into the base design of the user's interface.

Referring to FIGS. 15A-15D, which are representative of an image feedback control of the robot sub-system, and returning to FIGS. 1 and 14, the tracking system (i.e., Endoscout® tracking system) receives the tracking point coordinates from the Endoscout® sensor. The tracking point may be chosen manually based on the image from the camera. Once the neurosurgeon enters a command to move the tip of the robot closer to the target point (tumor), the data transformation unit (data processor) 62 calculates one (or several) centers of rotation 166 and actuates the corresponding actuating mechanism 42 operatively coupled to the joint(s) corresponding to the center(s) of rotation 166 to rotate respective links of the robot body towards the target point. Upon completion of the manipulation, the Endoscout® sensor 140 sends the new coordinates of the track point, as shown in FIG. 15B, to the Endoscout®Host PC 162 which provides this information to the MNHPC 160. The images on the MNHPC 160 are then updated in real-time for the neurosurgeon's use.

Since, as shown in FIG. 15B, the track point is still positioned far from the target point, the physician continues manipulation by entering further commands into the interface 38, i.e. MNH PC 160. After obtaining the coordinates for rotation center of the track point, the robot is moved further towards the target point as shown in FIG. 15C, and this process continues until the track point is aligned with the target point as shown in FIG. 15D. At this point, the end-effector of the robot is at the tumor location, and the physician may issue a command to initiate the surgery through the chosen modality by actuating the probes (end-effector) 150. The motion of the robot is envisioned to be automatic from the initial configuration to the final configuration. The neurosurgeon does not need to enter the points continuously. If the initial and final positions are identified, then an autonomous motion planning strategy is used while providing real-time images to the neurosurgeon.

It is important to note that in addition to an automated system where the neurosurgeon teleoperatively directs the robot to assume a particular configuration and carry out the treatment for tissue liquefaction, using one or another method, including, but not limited to the following: monopolar electrocautery, bi-polar electrocautery, Argon-Plasma coagulation, laser ablation, RF ablation, ultrasonic cavitation, etc., the subject system is provided with means enabling the physician to manually control the robot configuration by inputting a desired configuration of the robot on the screen by, for example, “clicking and dragging” the image of the end-effector presented on the virtual display while the system calculates the optimal way to reach the desired robot configuration.

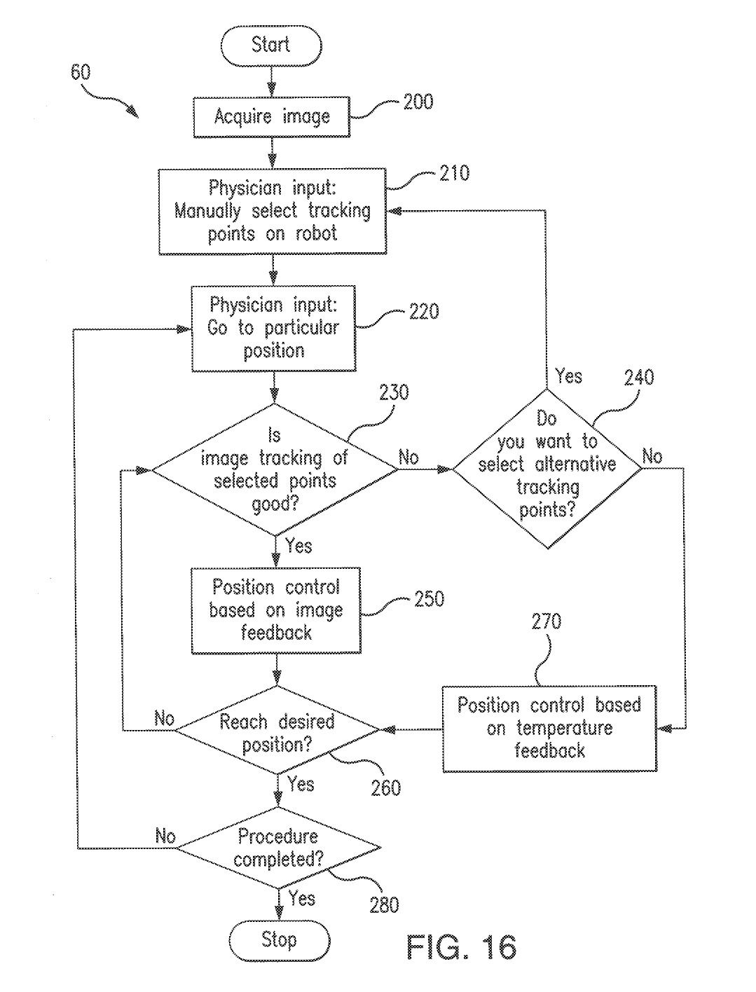

Referring to FIG. 16, representing the flow chart of the overall process, i.e., the user-MINIR interaction software 60 (shown in FIG. 1), the procedure starts at step 200 where the MR image is acquired and presented on the MNHPC 160.

The procedure is initiated with alignment of the robot joints so that the robot's configuration is straight at the start of the procedure, as shown in FIG. 6. This position is registered in the MR image. It is envisioned that a calibration routine may be performed as part of the MINIR operation. Further, in step 210, the neurosurgeon is prompted to enter commands, which may be entered by manually selecting tracking points on the robot. In further step 220, the physician is prompted to input the command “Go to Particular Position”.

Upon receiving the command of the neurosurgeon entered in step 220, the logic requests in step 230 as a decision whether the image of the selected tracking points is satisfactory. If the answer is negative, the logic flows to step 240 and requests whether the neurosurgeon desires to select alternative tracking points. If the neurosurgeon agrees, the logic loops to step 210, where the neurosurgeon can select alternative tracking points on the robot. If however, in step 230, the image tracking of the selected points is satisfactory, the procedure follows to step 250 to control position based on the image feedback as was detailed in previous paragraphs.

If in step 240 the neurosurgeon does not desire to select alternative tracking points, the procedure follows to block 270 and the control sub-system controls the position based on the temperature (or, alternatively, on motors rotary encoders' readings) feedback. At this point, the temperature data collected in real-time and stored during the robot operation, are used to determine the joint angle based on the collected temperature data.

From step 250, and/or alternatively from step 270, the process flows to logic block 260 where the system requests the neurosurgeon to determine whether the desired position has been reached. If the desired position has been reached, the logic flows to block 280 where the neurosurgeon is requested as to whether the procedure is completed. If the procedure is completed the process is terminated.

If however in step 260, the desired position has not been reached, the logic loops to the step 230 and prompts the neurosurgeon to answer whether the image tracking of the previously selected points is satisfactory.

In step 280, if the procedure is not completed, the logic returns to step 220, and prompts the neurosurgeon to input another particular position of interest.

The materials used for the robot sub-system parts are selected from the standpoint of minimal image distortion and corresponding high SNR (signal-to-noise ratio), which, in turn, may help with better localization of the robot body as well as the localization of the end-effector with respect to the tumor. In addition to plastics, MRI compatible metals may be used for the body of the robot, such as brass, titanium, etc. The robot body is enveloped into a medically inert plastic material 170 which smoothes the overall configuration of the robot to prevent direct contact between the moving parts of the robot and patient tissue in the intraoperative site in order to reduce trauma to the brain while the robot is being navigated.

The channels in plastic links 24 of the robot body 20 may be formed using a rapid prototyping. If metal links are used for the robot, then other approaches may be used for channel formations such as EDM (Electrical Discharge Machining). It may also be possible to machine the channels depending on the required tolerances. The links are envisioned to be made individually and then assembled together.

Upon performing the procedure, the robot sub-system may be disposed of. If the robot body 20 is made by rapid prototyping/inexpensive fabrication process, then the robot may be disposed along with the immediate quick-connect mechanism, except the SMA spring actuator sub-system and the routing box, which are positioned away from the operation site, and thus are not exposed to contact with a patient's tissues.

However, if the robot body is made of metal and cost of manufacturing is high, then the robot may be reused after sterilization while possibly only disposing the intermediate quick-connect mechanism 44.

Summarizing the description presented supra, the MINIR system is designed based on the following principles:
1. Since SMA actuators may cause some image distortion, in the present system, the SMA actuators designed to exert the required torque at each joint of the robot body through the tendon-driven mechanism, are removed from the MINIR body and are positioned outside the imaging system;
2. Hollow inner core design of the MINIR enhanced through tendons routing through the walls of MINIR links enables passage of the required wiring and suction and irrigation tubes inside the robot body;
3. A latching mechanism is provided which permits the quick-connect base of the MINIR to be latched in the cannula after the neurosurgeon advances the MINIR into the operation site to a required depth. This arrangement allows secure maintenance of the MINIR in position during the surgical procedure;
4. Unique tendon-driven mechanism has tendons routed through the body of the robot links for the most part. Tendons emerge very close to each joint axis as they transition from one link to the other. The advantage of this design is the ability to have substantially independent joint motion, since the tension in the tendon is prevented from causing a significant torque about the other joints which are not to be actuated. To enable the independent joint control, each joint needs to be individually controlled;
5. The intermediate quick-connect mechanism extends between the MINIR robot and the SMA spring actuators. The intermediate quick-connect mechanism is provided with a quick-connect feature at its both ends to be able to quickly connect/disconnect to/from the base of the robot body and the actuators side;
6. Integration of a commercially available Endoscout® tracking system (manufactured by Robin Medical, Inc.) with the robot body (one sensor at the tip of the robot and the other sensor at the distal end of the cannula) where the quick-connect mechanism is latched.
7. By using SMA springs with varying stiffness properties, a sufficient torque may be exerted at each joint to enable the robot motion in the workspace.
8. The system in question is envisioned to be equipped with custom design plug-and-play software platform that readily integrates to provide communication with the MINIR hardware and any MRI scanner, and further provides an interface for suitable interventional imaging manipulations;
9. MINIR may be adapted for communication with a number of imaging modalities. In one embodiment, it is specifically suited for MRI pulse sequence to enable communication with the MINIR for real-time tracking and surgical planning support.
10. MINIR is designed as a miniature multi-degree-of-freedom device;
11. To prevent the interference of SMA springs with the imaging, the cables (tendons) extending from one end of the quick-connect mechanism are routed outside the brain through small diameter plastic tubes (sheaths). The cables (tendons) on the other end of the intermediate quick-connect mechanism terminate at the quick-connect port where the SMA (or other type) actuators are located. With this design, after the procedure is completed, the entire device may be disposed leaving only the SMA spring actuators box (if that is the actuator of choice) and the routing box for Endoscout®, electrocautery, suction, and irrigation hardware in place for future use;
12. Different actuator strategies are envisioned for use in the MINIR. In one approach the tendons may be operatively coupled to the shaft of an MRI compatible Piezo LEGS rotary motors (manufactured by PiezoMotor, Uppsala, Sweden), for example, which can provide sufficient torque to move the various links of the MINIR robot through appropriate gearing and torque or motion amplification. In another approach, an individual gear and pulley system may be used for each direction motion of each individual joint with the tendons routed to an appropriate actuator. The choice of the actuators will depend on the required image quality in MRI.

Although this invention has been described in connection with specific forms and embodiments thereof, it will be appreciated that various modifications other than those discussed above may be resorted to without departing from the spirit or scope of the invention as defined in the appended claims. For example, functionally equivalent elements may be substituted for those specifically shown and described, certain features may be used independently of other features, and in certain cases, particular locations of the elements may be reversed or interposed, all without departing from the spirit or scope of the invention as defined in the appended claims.

1. Minimally Invasive Neurosurgical Intracranial Robot (MINIR) system, comprising:
a robot sub-system compatible with an imaging system and introduced in an intracranial area containing a target of interest;
a tracking sub-system operatively coupled to said robot sub-system and generating tracking information corresponding to said robot sub-system position;
an interface operatively coupled to said imaging system and said tracking sub-system to display substantially in real-time images of the intracranial area generated by said imaging system aligned with said tracking information, wherein said interface is further operatively interconnected between a user and said robot sub-system, and wherein the user applies commands to said interface to manipulate said robot sub-system based on said substantially in real-time images and said tracking information to reach said target of interest for an intended interaction therewith;
wherein said robot sub-system includes:
a robot body composed of a plurality of links and N revolute joints interconnecting respective of said plurality of links each to the other, wherein each of said N revolute joints is formed between respective adjacent links for rotational motion of each link with respect to the other about a corresponding rotational axis extending through said each revolute joint in substantially orthogonal relationship to a rotational axis of an adjacent revolute joint;
a tendon sub-system integrated with said robot body and containing N independent tendons routed through walls of said plurality of links, wherein each of said N tendons is operatively coupled to a respective one of said N revolute joints;
an actuator sub-system operatively coupled to said tendon sub-system, said actuator sub-system containing N independently operated actuating mechanisms, wherein each actuating mechanism is operatively coupled to a respective one of said N revolute joints through a respective one of said N tendons to independently control said respective revolute joint through controlling the motion of said respective tendon of said tendon sub-system; and
a control sub-system operatively coupled between said interface and said actuator sub-system;
wherein said control sub-system generates control signals responsive to the user's commands input via said interface and transmits said control signals to said actuator sub-system; and
wherein said actuator sub-system, responsive to said control signals received thereat, controls, through controlling the motion of at least one said respective tendon, the rotational motion of adjacent links of at least one said revolute joint, thereby steering said robot sub-system relative to said target of interest.

2. The system of claim 1, wherein said plurality of links include a tip link, a base link, and intermediate links interconnected between said tip and base links, and wherein said tip link includes an end-effector attached thereto.

3. The system of claim 2, further comprising an irrigation channel extending internally through said robot body between said tip link and an external irrigation hardware, wherein one end of said irrigation channel extends for interaction with said intracranial area.

4. The system of claim 2, further comprising a suction channel extending internally through said robot body between said tip link and an external suction hardware, wherein one end of said suction channel extends for interaction with said intracranial area.

5. The system of claim 2, wherein said end-effector is adapted for said intended interaction with said target of interest, and wherein said end-effector is electrically coupled to a end-effector hardware through wiring extending internally of said robot body.

6. The system of claim 5, wherein said end-effector is adapted for a tissue liquefaction at said target of interest.

7. The system of claim 5, wherein said end-effector operates in a mode selected from a group consisting of: monopolar electrocautery, bi-polar electrocautery, APC (Argon-Plasma Coagulation), laser ablation, radio-frequency ablation, and ultrasonic cavitation.

8. The system of claim 2, further including a flexible cannula insertable in a surgical corridor extended towards said intracranial area and configured to permit passage of said robot body therethrough.

9. The system of claim 8, wherein said tracking sub-system is integrated with said robot sub-system and operatively coupled to said interface and includes:
a first sensor integrated with said robot body and positioned at said tip link,
a data processing unit positioned externally of said intracranial area, and
wiring extending internally through said robot body between said first sensor and said data processing unit.

10. The system of claim 9, wherein said tracking sub-system further includes a second sensor positioned at a distal end of said flexible cannula.

11. The system of claim 8, wherein said flexible cannula is formed with a latching mechanism positioned at an internal wall of said flexible cannula at a distal end thereof, and wherein said latching mechanism is engageably compatible with said base link of said robot body to secure said base link to said flexible cannula at said distal end thereof.

12. The system of claim 11, wherein said latching mechanism includes a plurality of latches arranged circumferentially at said internal wall of said flexible cannula.

13. The system of claim 12, wherein said circumferentially arranged latches are positioned at a plurality of selected distances from an edge of said flexible cannula at said distal end thereof.

14. The system of claim 1, wherein said actuator sub-system includes N independently controlled SMA (Shape Memory Alloy) spring actuators, wherein each spring actuator is operatively coupled to said respective revolute joint via said respective independent tendon of said tendon sub-system.

15. The system of claim 14, wherein each of said N SMA spring actuators includes antagonistically coupled SMA springs.

16. The system of claim 10, further comprising a visual feedback sub-system coupled between at least one of said first and second sensors and said control sub-system.

17. The system of claim 15, further comprising electrical current source, wherein each of said SMA springs is independently coupled to said electrical current source to attain a corresponding temperature regime, thereby resulting in tension difference between a heated and unheated SMA springs.

18. The system of claim 17, further comprising a temperature based feedback sub-system coupled between said SMA springs and said control sub-system, said temperature based feedback sub-system acquires data on the temperature regime applied to a respective SMA spring and a corresponding rotational angle of a revolute joint affected by said respective SMA spring.

19. The system of claim 1, further including a first set of N gears secured in said base link, each of said gears in said first set thereof includes a respective pulley carrying therearound a respective one of said N tendons of said tendon sub-system.

20. The system of claim 19, further including a second set of N gears operatively coupled with said gears in said first set thereof, and an intermediate tendon sub-system including N intermediate tendons, wherein each intermediate tendon extends between a respective one of said gears in said second set thereof and a respective one of said N actuating mechanisms to independently control the motion of a corresponding one of said N tendons of said tendon sub-system, thereby controllably steering said robot sub-system.

21. The system of claim 20, wherein said N gears in said second set thereof are positioned at a single shaft attached to a base member, and
wherein said base member is removably secured to said base link.

22. The system of claim 21, further comprising an intermediate quick-connect mechanism having said base member with said second set of gears at one end thereof, and a third set of N gears positioned at another end thereof, wherein said each intermediate tendon extends therebetween.

23. The system of claim 22, further comprising:
a plurality of intermediate tendon routing pulleys and a plurality N of intermediate tendons coupled to said routing pulleys between said N actuating mechanisms and said third set of N gears, wherein said intermediate quick-connect mechanism is removably attached by said another end thereof to said routing pulleys and by said one end thereof to said base link.

24. The system of claim 1, wherein said control sub-system includes:
a data transformation unit receiving, at an input thereof, the user's commands, and computing corresponding control signals based on the position and configuration of the robot body, said control signals including coordinates of at least one center of rotation at said robot body and tracking path interpolation, said control signals being operatively applied to said actuator sub-system to control motion of at least one corresponding tendon in said tendon sub-system.

25. The system of claim 24, wherein said actuator sub-system further includes N motors, each operatively coupled to a respective one of said N revolute joints, and
wherein said control signals are applied to at least one of said N motors to actuate the same for controlling the motion of said at least one corresponding tendon in said tendon sub-systems.

26. A method for minimally invasive intracranial neurosurgery, comprising the steps of:
forming a surgical path towards an intracranial area containing a target of interest;
introducing a Minimally Invasive Neurosurgical Intracranial Robot (MINIR) device to said intracranial area through said surgical path;
wherein said MINIR device includes a robot body composed of a plurality of links interconnected at N revolute joints, wherein each one of said N revolute joints is formed between respective adjacent links from said plurality thereof for rotational motion of each link with respect to the other about a corresponding rotational axis extending through said each revolute joint in substantially orthogonal relationship to a rotational axis of an adjacent revolute joint,
a tendon sub-system integrated with said robot body and containing N independent tendons routed through walls of said plurality of links in a predetermined order, wherein each of said N tendons is operatively coupled to a respective one of said N revolute joints; and
a tracking sub-system having at least one sensor positioned in proximity to a tip of said robot body and generating information corresponding to a position of said tip of said robot body;
obtaining, substantially in real-time, images of said intracranial area containing the target of interest on a display of an user's interface;
aligning said tracking information acquired from said tracking sub-system and said in real-time images of said intracranial area on the display of the user's interface; and
receiving, through said interface, the user's commands to control said robot body position and configuration based on said tracking information and said in real-time images; and
responsive to the user's commands, calculating and operatively applying control signals to said tendon sub-system to control rotational motion of at least one respective revolute joint through controlling motion of at least one tendon is said tendon sub-system coupled to said respective revolute joint, thereby navigating said robot body relative to said target of interest.

27. The method of claim 26, further comprising the steps of:
operatively coupling a control sub-system between said tendon sub-system and said interface;
coupling an actuator sub-system between said control sub-system and said tendon sub-system, wherein said actuator sub-system includes N actuating mechanisms, each operatively coupled to a respective one of N independent tendons in said tendon sub-system; and
controlling the rotational motion of said at least one respective revolute joint through controlling the motion of said respective independent tendon by a respective actuating mechanism in correspondence to said control signals applied to said respective actuator mechanism.

28. The method of claim 26, further comprising the steps of:
routing an irrigation channel internally through a robot body between said tip link and an external irrigation hardware, and extending one end of said irrigation channel for interaction with said intracranial area.

29. The method of claim 26, further comprising the steps of:
routing a suction channel internally through said robot body between a tip link and an external suction hardware, and extending one end of said suction channel for interaction with said intracranial area.

30. The method of claim 26, further comprising the step of:
attaching an end-effector member to a tip link of said robot body, wherein said end-effector member is adapted for an intended interaction with said target of interest, and
operating said end-effector member in a mode selected from a group including: monopolar electrocautery, bi-polar electrocautery, APC (Argon-Plasma Coagulation), laser ablation, radio-frequency ablation, and ultrasonic cavitation.

31. The method of claim 26, further comprising the steps of:
inserting a flexible cannula in the surgical corridor, wherein said flexible cannula is configured to permit passage of said robot body therethrough, introducing said MINIR device to said intracranial area through said flexible cannula, and
securing said robot body at a distal end of said flexible cannula by a latching mechanism provided thereat.

32. The method of claim 27, wherein said real-time images are generated by an imaging system, selected from a group consisting of:
Magnetic Resonance Imaging (MRI) systems, a Computed Tomography (CT) imagining system, and an ultrasound imaging system.

33. The method of claim 27, wherein said actuator sub-system includes N independently controlled SMA (Shape Memory Alloy) spring actuators, wherein each spring actuator is operatively coupled to said respective revolute joint via said respective independent tendon of said tendon sub-system, and
wherein each of said N SMA spring actuators includes antagonistically coupled SMA springs.

34. The method of claim 27, further comprising step of:
coupling a visual feedback sub-system between said tracking sub-systems and said control sub-system.

35. The method of claim 33, further comprising the step of:
applying an electrical current to a respective one of said SMA antagonistically coupled springs to attain, in a controlled fashion, a corresponding temperature regime, thereby resulting in tension difference between a heated and unheated SMA antagonistically coupled springs.

36. The method of claim 35, further comprising the steps of:
coupling a temperature based feedback sub-system between said SMA antagonistically coupled springs and said control sub-system, and
acquiring data on the temperature regime applied to a respective SMA spring and a corresponding rotational angle of a revolute joint affected by said respective SMA spring.

37. The method of claim 32, further comprising the steps of:
positioning said actuator sub-system remotely from said imaging sub-system, and
connecting an intermediate quick-connect mechanism between said robot body and said actuator sub-system, wherein said intermediate quick-connect mechanism includes N intermediate tendons extending between said actuator system and said robot body.

38. The method of claim 27, further comprising the steps of:
receiving, at an input of said control sub-system, the user's commands, and
computing corresponding control signals based on the position and configuration of the robot body, where said control signals includes coordinates of at least one center of rotation at said robot body and tracking path interpolation, and
applying said control signals to said actuator sub-system to control motion of at least one corresponding tendon in said tendon sub-system.

39. The method of claim 27, further comprising the step of:
obtaining high-resolution diagnostic quality images of the intracranial area.
